(19) United States
(12) Patent Application Publication
(10) Pub. No.: US 2025/0210140 A1
(43) Pub. Date: Jun. 26, 2025

(54) MAPPING RESOLUTION USING SPATIAL INFORMATION OF SEQUENCED READS

(71) Applicant: Illumina, Inc., San Diego, CA (US)

(72) Inventors: James Han, San Carlos, CA (US); Taher Mun, West Chester, PA (US); Fabio Zanarello, Cambridge (GB); Mitchell A. Bekritsky, Decatur, GA (US); Arun Subramaniyan, San Diego, CA (US)

(21) Appl. No.: 18/984,835

(22) Filed: Dec. 17, 2024

Related U.S. Application Data
(60) Provisional application No. 63/614,066, filed on Dec. 22, 2023.

(51) Int. Cl. G16B 30/10; G16B 50/30

(52) U.S. Cl. CPC G16B 30/10; G16B 50/30

(57) ABSTRACT

Described are DNA sequencing systems and methods. Systems and methods may retrieve data comprising polynucleotide sequence reads and their spatial location on a sequencing substrate to determine spatially linked read pairs, identify a first polynucleotide sequence read with an alignment field indicating that the first polynucleotide sequence read ambiguously maps to two or more locations in the target polynucleotide sequence, map the first polynucleotide sequence read to a location in the target polynucleotide sequence when the first polynucleotide sequence read is spatially linked to a second polynucleotide sequence read having an alignment field indicating an unambiguous mapping, store an updated alignment field for the first polynucleotide sequence read in computer memory for the mapped nucleotide read if the first polynucleotide sequence read can be mapped to a location.

### CROSS-REFERENCE TO RELATED APPLICATIONS

This application claims priority to U.S. Provisional Application No. 63/614,066, filed Dec. 22, 2023, the content of which is incorporated by reference in its entirety.

### BACKGROUND

### Field

The present disclosure relates to DNA sequencing systems and methods. In particular, this disclosure relates to systems and methods for improving mapping of sequence reads to a target nucleic acid using spatial information of where the sequence read is positioned on a flow cell.

### Background

Many types of nucleic acid molecules, such as genomic DNA, are often too long to be directly sequenced using modern sequencing technologies. Library preparation is a step performed before genome sequencing to facilitate the sequencing process and ensure accurate and efficient analysis of the genomic DNA. Library preparation involves fragmenting the DNA into smaller, manageable pieces. This fragmentation can be achieved through physical or enzymatic methods. Reducing the length of genomic DNA by fragmenting the DNA can allow for more efficient sequencing and enables the reconstruction of the original genome during data analysis procedures. Library preparation may also involve attaching adapter sequences to the fragmented DNA. Adapters can contain specific sequences, such as primer or index sequences, that are recognized by the sequencing platforms and are used for sequencing the DNA fragments. These adapters provide priming sites and identification tags for use during the sequencing process.

Traditional nucleic acid sequencing methods, and several types of next-generation sequencing methods, use a shotgun approach to sequence DNA, such as genomic DNA, from a biological sample. As used herein the nucleic acids to be sequenced from a biological sample are called template nucleic acid molecules. Specifically, template nucleic acid molecules from a biological sample are first fragmented in solution into smaller pieces that are amenable to next-generation sequencing (NGS) methods on a flow cell. In NGS methods, the fragmented template nucleic acid molecules are reduced to fragments of approximately 300-500 nucleotide, each one of which results in a sequence read. Traditionally, read mapping relies on aligning these sequence reads to a reference genome, such as the genome assembly GRCh38 from the National Center for Biotechnology Information at the National Library of Medicine. Mapping a read from the template nucleic acid molecule may use an alignment process that identifies the best match between the sequence read and the reference genome based on sequence similarity and homology between the two sequences. However, this method often struggles in regions where a sequence read may align with multiple sites on the reference genome due to genetic repeats in the reference genome or low-complexity sequences. Incorrect alignments can introduce errors into downstream analyses and interpretations, such as variant calling, structural variant detection, and gene expression studies.

The process of ordering the sequence reads to arrive at the correct nucleotide sequence of the original template nucleic acid molecule from the biological sample is generally referred to as “assembly.” Assembly processes can be computationally intensive and time-consuming. In addition, sequence and assembly errors can become a problem depending upon the sequencing methodology used and the quality of the nucleic acid molecule samples under evaluation.

### SUMMARY

This disclosure describes a novel method of disambiguating sequence reads coming from a template nucleic acid molecule that align to multiple locations in a reference genome. This is achieved by analyzing sequence reads which are located near each other on a flow cell, due to the discovery that it is more likely sequences located near each other on the flow cell were derived from the same template nucleic acid molecule. Embodiments relate to first determining an “anchor” sequence read which aligns to the reference genome with a high alignment score. The disclosure provides for determining sequence reads that are located near to an anchor sequence read on a flow cell and thus may be linked to the anchor sequence read such that there is a higher probability that the sequence read actually aligns to the reference genome at a position adjacent to the anchor sequence read. Unlike traditional methods which may discard, or erroneously use, sequence reads which are found to align to multiple locations on a reference genome, this approach uses the location information from the flow cell to link sequence reads having multiple alignments to an anchor sequence read having a known alignment to a specific location in the genome. This provides a targeted approach to determine the correct alignment of short sequence reads which may initially have multiple possible alignments to the reference genome and provide an accurate mapping of the sequence reads to the reference genome for final assembly of the final sequence of the target nucleic acid molecule being analyzed.

The alignment of sequence reads is an important starting point for genomics analysis, providing a basis for variant detection, functional annotation, and other downstream analyses. When a sequence read aligns to multiple regions of a reference genome, determining the most accurate placement becomes a challenge.

In some embodiments, the techniques described herein relate to a system for updating an alignment record of polynucleotide sequence reads from a target polynucleotide sequence including: at least one processor; and a non-transitory computer readable medium including instructions that, when executed by the at least one processor, cause the system to: retrieve data including polynucleotide sequence reads and their spatial location on a sequencing substrate to determine spatially linked read pairs; identify a first polynucleotide sequence read with an alignment field indicating that the first polynucleotide sequence read ambiguously maps to two or more locations in the target polynucleotide sequence; map the first polynucleotide sequence read to a location in the target polynucleotide sequence when the first polynucleotide sequence read is spatially linked to a second polynucleotide sequence read having an alignment field indicating an unambiguous mapping; store an updated alignment field for the first polynucleotide sequence read in computer memory for the mapped nucleotide read if the first polynucleotide sequence read can be mapped to a location.

In some embodiments, the techniques described herein relate to a method for improving mapping resolution for a polynucleotide sequence including: retrieving data including polynucleotide sequence reads and their spatial location on a sequencing substrate to determine spatially linked read pairs; identifying a first polynucleotide sequence read with an alignment field indicating that the first polynucleotide sequence read ambiguously maps to two or more locations in the target polynucleotide sequence; mapping the first polynucleotide sequence read to a location in the target polynucleotide sequence when the first polynucleotide sequence read is spatially linked to a second polynucleotide sequence read having an alignment field indicating an unambiguous mapping; and storing an updated alignment field for the first polynucleotide sequence read in computer memory for the mapped nucleotide read if the first polynucleotide sequence read can be mapped to a location.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear. While the disclosure has been illustrated and described in detail in the drawings and foregoing description, such illustration and description are to be considered illustrative or exemplary and not restrictive. The disclosure is not limited to the disclosed embodiments. Variations to the disclosed embodiments can be understood and effected by those skilled in the art in practicing the claimed disclosure, from a study of the drawings, the disclosure and the appended claims.

FIG. 1 schematically illustrates a non-limiting example of a solid support which can perform embodiments of the disclosed sequencing technology.

FIG. 2 shows a flowchart of an example method for improving mapping resolution using spatial information of sequenced reads.

FIG. 3 presents an iterative method designed to refine alignments in sequencing data by harnessing spatial information.

FIG. 4 is a bar chart that displays the performance of various sequencing technologies and methods in detecting single nucleotide variants (SNVs) across the genome.

FIG. 5 is a bar chart illustrating the genome-wide small variant performance across different sequencing technologies: IDPF, ICLR, and two versions of example methods (70× and 140× on NextSeq 2000).

FIG. 6 is a line graph showing the relationship between deduplicated linked read coverage and combined false positives and negatives (FP+FN) for germline sequencing.

FIG. 7 illustrates two panels and presents a visualization focusing on the RHCE gene, which encodes the Rh blood group, commonly known as Rh positive or Rh negative.

FIG. 8 illustrates two panels and presents a visualization focusing on the performance of the disclosed methods towards OTOA mutations that are associated with AR non-syndromic deafness.

FIG. 9 illustrates two panels and presents a visualization focusing on a depiction of the sequencing challenges surrounding the PDPK1 gene and showing the results of the disclosed methods.

FIG. 10 is a visualization which compares the performance of two sequencing assembly technologies, a comparative assembly technology and the example methods, in sequencing and detecting deletions (represented as “DEL1” and “DEL2”) in a reference genome.

FIG. 11 is a diagram of an exemplary computing system 1400 that may be used in connection with an illustrative sequencing system.

### DETAILED DESCRIPTION

All patents, applications, published applications and other publications referred to herein are incorporated herein by reference to the referenced material and in their entireties. If a term or phrase is used herein in a way that is contrary to or otherwise inconsistent with a definition set forth in the patents, applications, published applications and other publications that are herein incorporated by reference, the use herein prevails over the definition that is incorporated herein by reference.

Embodiments relate to systems and methods for improving mapping of sequence reads to a reference genome, by linking ambiguously mapped sequence reads to sequence reads which have a high quality of mapping to a reference genome. The sequence reads may be derived from a fragmented template nucleic acid molecule, such as a fragmented genomic DNA molecule. The data from a fragmented genomic DNA molecule may be used to determine the genotype and variants found in the genomic DNA. These embodiments help to overcome many of the traditional limitations of short read sequencing in detecting variants, and particularly, structural variants (SVs) in the template nucleic acid molecule. As discussed above, in some next generation sequencing (NGS) systems, fragments of relatively long DNA, such as those derived from genomic DNA from a biological source, are fragmented to create shorter template nucleic acid molecule fragments which can be sequenced in a single read on a flow cell or other process. The fragmenting process creates shorter nucleic acid fragments which land on and bind to a flow cell or other type of solid substrate in some embodiments. The spatial location of where each nucleic acid fragment binds on the solid substrate was found to correlate with the template nucleic acid molecule fragment's position in the original template nucleic acid molecule from which the fragment was derived. For example, fragments which came from the same portion of a template genomic DNA were found to bind closer together to one another on the flow cell as compared to fragments which came from different portions of the genomic DNA molecule.

In some embodiments, methods and systems according to the disclosure may be directed to resolving ambiguous alignments, particularly near segmental duplications, which is typically difficult due to the high sequence similarity in these regions. A method to rescue reads from such ambiguous alignments could involve an iterative process that starts at the ends of segmental duplication copies. For example, the method may proceed by first selecting anchor reads located at the edges of a structural variant, such as, for example, a segmental duplication. By accurately aligning high mapping quality reads in these regions first, this information can be used to resolve more complex alignments towards the middle of the duplications where the mapping quality may be lower. In some embodiments, a high mapping quality (or in some case a high alignment quality score) may be a score of, for example, 30 in arbitrary units consistent with a phred score. Once a set of reads is disambiguated at the end of a duplicated region of a genome, the newly disambiguated reads with a high mapping quality may serve as anchor reads for other more ambiguous reads which link to these anchor reads. These linked reads can then act as new anchor reads which are then used to guide the alignment of other adjacent sequence reads that originate from the same long template fragment but were found to map ambiguously within the segmental duplication location on the genome. As the process iterates, each cycle uses any newly disambiguated reads to clarify the placement of other ambiguous reads near the duplication, propagating certainty and mapping quality inward from the ends of the duplication segments towards the center. This chain effect leverages the spatial relationship of sequence reads derived from the same template nucleic acid molecule, gradually reducing ambiguity and improving alignment confidence across the region.

In many NGS systems, the nucleic acid fragments are bound to a flow cell and are then subjected to amplification reactions to generate clusters of clonal copies of the bound fragment. Accordingly, if two clusters of nucleic acid fragments on a flow cell are close together spatially on a flow cell, it was discovered that it is more likely that the nucleic acid fragments making up each cluster are also more likely to have come from the same location on the original template nucleic acid molecule. Of course, it's also possible that unrelated nucleic acid fragments may bind near each other on the flow cell, which can lead to an uncertainty in the probability that adjacent clusters originated from the same template nucleic acid molecule.

A number of factors can affect the probability that unrelated nucleic acid fragments would form clusters near each other on a flow cell, and these factors may change based on a variety of experimental conditions. Embodiments of the invention provide a statistical method for calculating the probability that two sequence reads coming from two clusters of nucleic acid fragments are linked, such that on a flow cell the two sequence reads were derived from the template nucleic acid molecule.

Some embodiments provide for establishing the quality of a link between two or more pairs of nucleic acid reads (“read pairs”) on a flow cell. The “link” as discussed herein is the probability that two pairs of sequence reads on a sequencing flow cell are derived from the same original nucleic acid molecule. In some embodiments, the link between two pairs of reads on a sequencing flow cell does not require a quantifiable metric to determine the quality of the link between two reads.

Some embodiments of the invention relate to systems and methods for sequencing target nucleic acids by fragmenting the target nucleic acid molecules and distributing the resulting fragments onto a solid substate that is a flow cell. As the fragments are distributed along the flow cell, they bind capture primers and are then amplified by bridge amplification to create clusters by well-known technologies, such as those provided by Illumina Inc. (San Diego, CA). As described above, nucleic acid fragments which were derived from the same template nucleic acid molecule are more likely to bind to the flow cell in spatially adjacent positions as compared to fragments that are from different template nucleic acid molecules, particularly when the fragmentation is performed directly on the flow cell using immobilized transposome complexes on the surface of the flow cell. This spatial information can be used to help guide assembly and variant calling of the original template nucleic acid molecule, as will be described in more detail below.

For example, in some embodiments, transposome complexes are bound to the surface of a flow cell. The transposome complexes include a transposase and a first polynucleotide having end sequences which can be used to fragment the template nucleic acid molecule and insert into each fragment an end sequence or tag which can be used to bind to capture probes located on the substrate. The method can include contacting the transposome complexes with the fragments of the template nucleic acid molecule under conditions to fragment the template nucleic acid molecule and add capture sequences to the ends of each fragment. In some embodiments, the capture sequences include P5 or P7 sequences as provided by Illumina, Inc. In some embodiments, the complexed strand and transposome is in solution, and is then brought towards a substrate and immobilized thereon. In some embodiments, prior to immobilization of the transposome complexes on the substrate, one or more of the transposome complexes bind the template nucleic acid molecules in solution. In this embodiment, the transposome complexes in solution become immobilized to the substrate.

Once the template nucleic acid molecule fragments have been bound to substrate, the bound fragments can be amplified through bridge amplification to form a plurality of nucleic acid clusters on the substrate. The location of each cluster on the flow cell can then be determined before, during or after performing sequencing by synthesis reactions (SBS) to obtain the nucleotide sequence of each fragment located in each cluster to determine the sequence read of that cluster. Once the nucleotide sequence of each cluster has been determined, the method can start to map those reads to a reference genome to determine the position of each sequence read in the original template nucleic acid molecule from which the read originated.

In some embodiments, the mapping process takes into account the spatial location of each cluster on the flow cell, such that clusters which are closer to each other on the flow cell are more likely to have originated near each other in the template nucleic acid molecule. In some embodiments, the library preparation steps are performed on the flow cell, which may reduce the complexity and the amount of equipment required for the systems. Furthermore, by mapping the sequence reads to a reference genome using the spatial information accompanying each cluster, the method performs more accurate mapping operations as compared to methods that do not take the spatial location of each cluster into account during the mapping process. Therefore, spatial information that includes the relative distance between various clusters on a flow cell is leveraged to increase the accuracy of read mapping by helping properly assign a particular sequence read to its correct position on the reference genome, as compared to prior systems which could not properly map sequence reads that would map to multiple possible positions (“multi-mapped reads”). For example, in prior systems, multi-mapped reads which mapped to several possible positions on a reference genome may have been discarded as unmappable. With current embodiments, the confidence of a read pair's mapping to a reference genome is increased by linking a sequence read which is initially ambiguously mapped, or multi-mapped, to a sequence read which has a high mapping quality score, based on how adjacent each of their clusters are to one another on a flow cell. This may improve the alignment information and quality of information used in certain genomic analysis applications including, but not limited to, variant calling.

Embodiments may use a linking quality score, which is a numerical representation that quantifies the reliability of a link between two read pairs. This can be one measure of the strength of a correlation between two sequence reads, such as an ambiguously mapped read, and an anchor read with their genomic location on a template nucleic acid molecule. A high linking quality score means that two sequence reads are likely to be located near each other on the template nucleic acid molecule, and conversely a low linking quality score means that two sequence reads are not likely to be located near each other on the template nucleic acid molecule. This measure of quality of has implications for various downstream applications including mapping, alignment, and variant calling. This link quality score not only enables the filtration of potentially erroneous reads but also aids in identifying high-quality links between two sequence reads on a flow cell. As a result, the downstream processes become more efficient while also minimizing the computational memory required.

Another relevant aspect is the use of long range connectivity information to confirm or identify structural variants within a template nucleic acid molecule. The nature of structural variants themselves makes them difficult to detect with short reads, such as the 300-500 base pair reads found in many NGS applications. Structural variants include deletions, insertions, duplications, inversions, and translocations that can range in size from a few base pairs to several megabases. When the size of the variant is near to, or exceeds, the length of the short reads, it becomes problematic to assign the sequence read to a particular site which contains the variant. In addition, because many short read sequencing systems map the sequence read to a reference genome, if a structural variant is present in the template nucleic acid molecule, the short read from that structural variant region may not align properly or at all to the reference genome. For example, the short read may come from a template nucleic acid molecule which has a structural variant of a kilobase insertion in the genome. The sequence read from the template nucleic acid molecule which corresponds to the position of the kilobase insertion will align that sequence read to the original position of that insertion in the reference genome, and not its new position as an insertion, since the sequence read will be too short to cover areas outside of the insertion. Additionally, repetitive regions in the genome exacerbate the challenges posed by short read sequencing. A significant portion of the human genome is composed of repetitive sequences. If a structural variant occurs within or near these repetitive regions, short reads may not provide unique alignment information. Determining the exact placement and context of such reads is challenging, leading to ambiguities in SV detection.

The introduction of long-range connectivity information in short read sequencing serves as an intermediary solution that bridges the gap between traditional short read sequencing and long-read sequencing in the context of structural variant detection. Firstly, the methods of the disclosure allow for the grouping of short reads that originate from the same, longer DNA molecule. This means that even if individual reads might be too short to span an entire structural variant, the collective information from a group of short reads can provide context about larger regions of the genome. When short reads are associated within a longer original template nucleic acid molecule, sequencing methods gain insight into regions of the genome much larger than the individual read lengths, thereby aiding in SV detection.

Secondly, the long-range connectivity information aids in resolving the correct sequences of repetitive regions of the genome. By associating such short reads with others from a known anchor read or fragment, one can more confidently place these reads in their correct genomic context, reducing ambiguity and increasing the accuracy of SV detection. Additionally, having this extended context helps in the accurate reconstruction of the genomic landscape. This is particularly beneficial when dealing with complex structural variants or regions with multiple variants close together. Traditional short read methods might struggle to differentiate between such scenarios, but the added context from long-range connectivity can help disambiguate such scenarios.

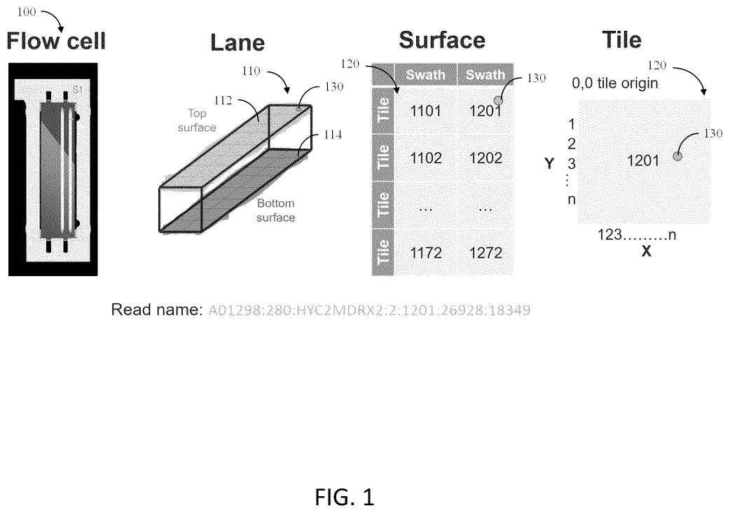

Referring now to FIG. 1, in some embodiments, a flow cell 100 provides spatial information of read pairs and includes a plurality of lanes 110. Each lane 110 includes a plurality of surfaces, including a top surface 112 and a bottom surface 114. By way of example, if the sequence reads being compared are on opposite surfaces, the distance between them is considered infinite because the assumption is that they cannot be linked. Template nucleic acid molecules which are flowed over a surface are fragmented by transposomes bound to each surface, so it would not be possible for the same template nucleic acid molecule to flow and be fragmented on the top and the bottom surface. Thus, two sequences reads from the top and bottom surfaces are assumed to not be linked since they are so unlikely to have been derived from the same template nucleic acid molecule. Note, however, that in some embodiments, it is possible that reads from different surfaces could be linked, especially as the size of the input template DNA molecule increases.

In some embodiments, each surface is subdivided into a plurality of tiles 120. As shown, a cluster 130 may be located on a tile 120 that is designated as 1201. This designation serves as an illustrative example only and is not limited to the alphanumeric characters shown in the figure. In some embodiments, the tile 120 includes two-dimensional X-Y coordinates as shown to provide the spatial information between clusters. In some embodiments, the X-Y coordinates may be derived from information stored in a FASTQ file. In some embodiments, X-Y coordinates may be stored in or derived from a BCL (Base Call) file, which is a binary file format commonly associated with next-generation sequencing (NGS) platforms.

In some embodiments, the subdivision of the surface into tiles 120 is an artificial separation so that the surface of the flow cell is not separated into physical tiles, but instead the images captured by a camera can be segmented into tiles. As shown, the tiles 120 are subdivided into swaths, which roughly correspond to a pixel width of a camera used to capture images of the flow cell. In some embodiments, the tile 120 denotes the size of an image that can be captured by the camera. In some embodiments, the X-Y coordinates are pixel values. In some embodiments, 1 unit of a tile 120 can be approximated to be 1/10^{th} of a pixel. A physical separation is contemplated in some embodiments where the tile can have physical barriers, wells, and other structures which separate one portion of the flow cell from another portion of the flow cell. In some embodiments, spatial information, including X-Y coordinates, for clusters such as cluster 130 are obtained by a camera that processes the pixel value of the digital image.

One experiment that may provide spatial information on sequenced reads may be performed on a substrate having transposome complexes immobilized thereon. A transposome complex may include a transposase and a first polynucleotide including an end sequence and a first tag in some embodiments. The sequencing experiment may proceed by contacting the transposome complexes with target polynucleotides under conditions to fragment the target polynucleotides. The fragmented target polynucleotides may then be amplified to form a plurality of nucleic acid clusters on the substrate. The plurality of nucleic acid clusters on the substrate are microscopically observable and their location data may be recorded. After the location information has been obtained, then the nucleic acid sequence reads of the fragmented nucleic acids may be sequenced and the corresponding location data may be stored.

In some embodiments, a functional definition of “near” indicates that the sequence reads originate from the original template. Variably this may mean that near means within a threshold distance of 10,000 nm, 5,000 nm., 3,000 nm., 2,000 nm, and 1,000 nm. In some embodiments, nearby may mean within a certain number of proximate wells. For example, the number of wells between clusters may be much greater than 50, than 100, or than 200 wells. In some embodiments, nearby may depend on x/y direction as the diffusion pattern may not be uniform after fragmentation. For example, the links may form an oval pattern on the flow cell.

Described herein are systems and methods of establishing link quality scores for the links determined between read pairs based on spatial information obtained on the flow cell. This spatial information may be, for example, the geographical coordinates of the cluster which contains a particular read on the flow cell. The spatial information may include a location of a well on a substrate in one embodiment. To establish these spatial links between two reads, in one embodiment two thresholds are used. The first is the spatial distance threshold, which represents the physical distance between two reads on the flow cell.

In some embodiments, the spatial distance may be measured in nanometers. In some embodiments, the spatial distance may be measured in a unit of length relative to the flow cell. For example, a flow cell unit may be relative to the size and/or spacing of patterned clusters on a flow cell. In some embodiments, two differently patterned flow cells may have different absolute units of length due to different density of clusters on the surface. In some embodiments, the spatial distance may be an absolute unit of length, or any other unit of length consistent with the disclosure. In some embodiments, the spatial distance may be included in a FASTQ file, which generally is a text file that contains the sequence data from the clusters that pass filter on a flow cell. FASTQ files can be used as sequence input for alignment and other secondary analysis software.

The second threshold is a genomic distance threshold, representing the distance between the two reads on the genome after mapping. In some embodiments, a genomic distance may be based on a reference genome. In some embodiments, other methods may use distance in a sample genome. An empirical method for establishing thresholds will vary widely between experimental conditions. This disclosure provides for methods to attach a link quality score to a link as a factor of the spatial and genomic distance between two potentially linked reads. As described in more detail below, one method of determining the quality of a link between two reads is to estimate the null distribution of pairwise read pairs. This null distribution can provide the basis for calculating the “false discovery rate”, which can then be used as a proxy for the link quality score of the link.

A linking quality score is defined as a numerical representation that quantifies the reliability of a link between two read pairs. This score may be calculated using multiple metrics that contribute to the quality of the link, and the linking quality score may serve as a composite measure that simplifies complex relationships into a single, easily interpretable value.

The disclosure outlines a method to enhance mapping efficiency by examining the connections between read pairs on a flow cell's surface. A notable aspect of this method is the optional use of a link quality score, which aids in determining the linkage between read pairs. This linking quality score may provide a basis for comparison or decision-making. For example, a high linking quality score between two read pairs might indicate that two reads are highly likely to originate from the same portion of a template nucleic acid molecule, and thus should be paired for further analysis, but also that the conditions used to generate that link may be tuned and evaluated on the basis of the scores. Consistent with the disclosure, various factors may be considered for calculating the link quality score. These include the false discovery rate, a metric for type II error, a weighted average of different metrics, and the use of a machine learning model designed to predict link quality from multiple features. The linking quality score aims to encapsulate diverse considerations into a single number representing a link's overall “quality,” thereby facilitating quantitative analysis.

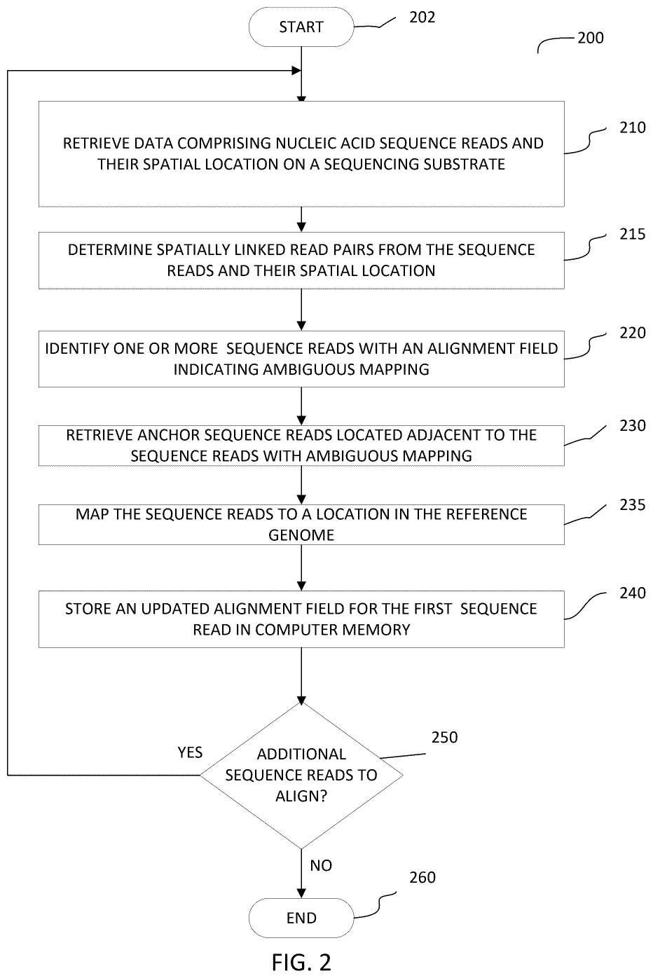

FIG. 2 is a flowchart of an example method 200 for retrieving sequence read data and then updating an alignment field in a computer memory for that sequence read based on the location of the sequence read on the flow cell. The process of obtaining sequence reads from a flow cell that contains clusters of fragments of a template nucleic acid molecule—while ensuring that fragments located near each other in the original template nucleic acid have a higher probability of being proximate on the flow cell—may entail the following steps starting at step 202.

As a preliminary step, a NGS process is run to generate data comprising the nucleotide sequence read of each fragment in a cluster on the sequencing substrate and their respective spatial positions on the sequencing substrate, such as a flow cell. Note that the method illustrated in FIG. 2 may be performed in real time, with a real time processing unit, or may be performed after sequencing, during a post processing analysis on a local or remote processor. For example, at step 210, a system may retrieve data comprising polynucleotide sequence reads and their spatial location on a sequencing substrate, such as the pixel(s) where the sequences were imaged or location of a nanowell where a cluster of fragments may have been amplified. The sequencing substrate may be a platform such as a flow cell, chip, or any other sequencing medium with an ability to provide the sequence data and also the relative physical positions of each read on the substrate.

The process 200 then moves to a step 215 to use the retrieved data to determine spatially linked read pairs. For example, the process 200 may calculate the reads which are within a specific distance from one another to determine that two read pairs are spatially linked. The process 200, for example, may determine that sequence reads within a distance of 10 nanometers from each other on the sequencing substrate are spatially linked. The disclosure provides several methods for evaluating this proximity and then determining spatially linked read pairs. These linked read pairs offer an additional layer of information, which can be used in parallel with the sequencing information to align and map the sequence reads. In some embodiments, the step of determining spatially linked read pairs may include determining the link quality of the links between the spatially linked read pairs. In some embodiments, spatially linked read pairs are read pairs with link that have a link quality score above a threshold value.

After obtaining the spatially linked read pairs, the method may proceed to a step 220 to identify specific sequence reads that have been ambiguously mapped to the target sequence. In general, this step scans the data corresponding to the sequence reads and reads the alignment fields of the sequenced reads to determine those reads without a clear, singular alignment to the reference genome. The alignment field is associated with each sequence read, and stores the information regarding the mapping of the sequence read to a particular position on the reference genome. For example, some alignment files may contain multiple potential mapped positions on the reference genome, creating ambiguity regarding the correct mapping assignment for that sequence read. In some cases, the alignment field may contain an alignment score below a threshold confidence level. An alignment field may also comprise multiple alignment scores for a sequence read that aligns to more than one location, where each putative alignment has an alignment score. An alignment field may also be a flag indicating that the alignment is suspect based on some indication that the alignment may be inaccurate.

Having identified a read with ambiguous mapping, the method may proceed to step 230 to retrieve anchor sequence reads located adjacent to any sequence reads which have ambiguous mapping. In some embodiments, retrieving anchor sequence reads located adjacent to ambiguous may include retrieving anchor reads that have similar coordinates on the flow cell. If any of the ambiguously mapped reads (the first polynucleotide sequence read) is spatially linked to an anchor read (the second polynucleotide sequence read) that has a clear and unambiguous alignment with a high mapping quality, this linkage can guide the mapping of the first ambiguously mapped read. The rationale is that spatially linked reads, due to their proximity on the substrate, have a high chance of originating from adjacent or nearby regions in the genome. Thus, the anchor read can provide context to accurately place the first, ambiguously mapped read to its proper position on the reference genome. Once the process 200 has determined if any anchor sequence reads are linked to ambiguously mapped reads, the process 200 can move to a step 235 to map the sequence read to a location in the reference genome. The method may selectively update the mapping when the first sequence read is spatially linked to a second polynucleotide sequence read having an alignment field indicating an unambiguous mapping, such as with an anchor sequence.

Once the first polynucleotide sequence read has been accurately mapped using the spatial linkage information, its alignment field may be updated at a step 240 to reflect the new mapped location of the sequence read in the reference genome. The revised alignment data may be stored in the alignment field in a computer memory, ensuring that all subsequent analyses or data retrievals utilize this enhanced, clarified mapping. In some embodiments, the alignment may be updated even if some ambiguous mappings remain. In some embodiments, the alignment field is only updated and stored if the first polynucleotide sequence read can be mapped to a location as shown in step 240. In some embodiments, ambiguously mapped reads may be reads aligning with a mapping quality of zero. In some embodiments, ambiguously mapped reads may refer to reads with a mapping score of below a mapping quality threshold. In some embodiments, ambiguously mapped reads may be any read that maps to more than one location on the reference genome.

In some embodiments, after a disambiguated sequence is aligned and the new alignment is stored in memory, the process may move to a decision step 250 to determine if any additional sequence reads are to be analyzed. If additional read pairs are left unmapped, or there is any other indication that there would be new high confidence anchor reads, the process 200 may return to step 210 to retrieve additional data on the sequence reads. However, in some embodiments, only the sequence reads that received updated alignment information may be selected. Accordingly, if the process is repeated, the entire file might not need to be reprocessed. If there is no further need to detect additional structural variants, the method conclude at end step 260.

Consistent with the disclosure, the genomic data referenced in the previous steps may be obtained by various methods, whether indirectly from databases, or pre-processed information, or from a sequencing system and any associated raw data. For example, one way to acquire genomic information referenced in step 210, may be by retrieving it from local or remote databases. These databases may store genetic data from various sources, including genomes, genes, sequences, and annotations. In some cases, genomic information may be pre-processed and shared directly. This pre-processed data could include aligned reads, variant calls, or other specific genomic analyses.

In bioinformatics, when referring to the alignment field, it is often in the context of a Binary Alignment/Map (“BAM”) or Sequence Alignment/Map (“SAM”) format, which are standard formats for storing large nucleotide sequence alignments. However, it should be realized that this disclosure considers alignment fields that include fields other than those in a BAM or SAM format. By way of a non-limiting example, each line of a SAM file represents a read and its alignment to a reference, with various fields describing the properties of this alignment. Some key fields of a SAM file include FLAG: Bitwise FLAG; RNAME: Reference sequence NAME; POS: 1-based leftmost mapping POSition; MAPQ: MAPping Quality; CIGAR: CIGAR string; RNEXT: Reference name of the mate/next read; PNEXT: Position of the mate/next read; TLEN: Observed Template LENgth; SEQ: Segment SEQuence; and QUAL: ASCII of Phred-scaled base QUALity+33.

Of these alignment fields, several may be used to indicate an uncertainty in an alignment. For example, if a certain chromosome is known to be difficult to align, then any of the alignment fields that correlate the sequence to that chromosome may be used as an alignment field that indicates that the sequence alignment is ambiguous. In some embodiments, the FLAG field may be used to indicate alignment, which is a bitwise representation of various properties of the read and its alignment. In some embodiments, a MAPQ field, which represents the mapping quality (and can be seen as a score) may be used. As an example, an alignment in SAM format may read as follows:

READ123 73 chr1 130 30 9M1I1M=151 21 AGTCAGTCA*NM:i:1.

In this example: READ123 is the name of the read. 73 is the FLAG, indicating the read is paired in sequencing, the first in pair, and the mate is in reverse orientation. chr1 is the reference sequence name. 130 is the starting position of the alignment. 30 is the MAPQ score, indicating a reasonably high confidence in the alignment. 9M1I1M is the CIGAR string, suggesting the read has 9 matches, 1 insertion, and then 1 match to the reference. In this context, the MAPQ score can be seen as the “alignment score”, while the FLAG provides various flags related to the read and its mate.

Some embodiments are directed to a system for updating an alignment record of polynucleotide sequence reads from a target polynucleotide sequence including: at least one processor; and a non-transitory computer readable medium including instructions that, when executed by the at least one processor, cause the system to: retrieve data including polynucleotide sequence reads and their spatial location on a sequencing substrate to determine spatially linked read pairs; identify a first polynucleotide sequence read with an alignment field indicating that the first polynucleotide sequence read ambiguously maps to two or more locations in the target polynucleotide sequence; map the first polynucleotide sequence read to a location in the target polynucleotide sequence when the first polynucleotide sequence read is spatially linked to a second polynucleotide sequence read having an alignment field indicating an unambiguous mapping; store an updated alignment field for the first polynucleotide sequence read in computer memory for the mapped nucleotide read if the first polynucleotide sequence read can be mapped to a location.

In some embodiments, systems and methods may include using an alignment field indicating that the first polynucleotide sequence read ambiguously maps to two or more locations is an alignment score. In some embodiments, the alignment field indicating an unambiguous mapping is an alignment score. In some embodiments, an ambiguous alignment occurs when the alignment score is below a first threshold value. In some embodiments, the first threshold value is a MAPQ score of approximately zero. The first threshold value may be, for example any of a MAPQ score of 0, 1, 2, 3, 4, 5, 10, 11, 12, 13, 14, and 15.

In some embodiments, a high confidence read, such as an anchor read may occur when an alignment score is above a second threshold value. In some embodiments, the second threshold value is a MAPQ score of more than 10. The second threshold value may be any of a MAPQ score of 10, 11, 12, 13, 14, 15, 16, 17, 18, 19, 20, 25, and 30. Similarly, in some embodiments, an updated alignment field may include a MAPQ score of more than 10, or also any of or above the values of the second threshold. In some embodiments, an updated alignment field is mapping quality, where a mapping quality below a threshold mapping quality score indicates ambiguity.

In some embodiments, the mapping quality is proportional to the difference in read pair alignment scores between a first and a second-best scoring alignment. In some embodiments, the updated alignment field is generated by an alignment process based on alignment of the first and second polynucleotide sequences to a reference genome. In some embodiments, the alignment process generates alignment scores for secondary alignments above a threshold alignment score. In some embodiments, the updated alignment score is a percentage of the alignment score corresponding to the primary alignment. In some embodiments, the updated alignment score is calculated based on a mapping quality. In some embodiments, the updated alignment score is based on a linking quality score.

In some embodiments, the alignment field indicating that the first polynucleotide sequence read ambiguously maps to two or more locations is a Boolean tag. In some embodiments, the Boolean tag corresponds to a likelihood above a third threshold value that the polynucleotide sequence read has an alignment property. In some embodiments, the alignment property may be at least one of chromosome, position, mapping quality and a tag indicating link information. In some embodiments, the Boolean tag is at least one of chromosome, position, mapping quality and a tag indicating link information. In some embodiments, the link information includes at least one of a number of links, a link quality, and any alignments of the sequence reads that are linked to one another.

In some embodiments, instructions to map the nucleotide read to a single location may be implemented during an alignment processing step. In some embodiments, mapping the nucleotide read to a single location may be implemented as an alignment post-processing step.

One method for identifying an ambiguous mapping includes filtering the sequence reads for sequence reads that map to two or more locations in the target polynucleotide sequence. Note that in some embodiments, many sequence reads may have a primary alignment with high confidence and a secondary alignment with low confidence such that the sequence alignment is not ambiguous. In some embodiments, each candidate alignment of an ambiguously mapped read (for example, candidates may align with a mapping quality of zero), high mapping quality alignments (with mapping quality at least 10) may be retrieved if nearby in the flow cell (in terms of flow cell coordinates) as well as nearby in the genome (in terms of genomic coordinates). These nearby high mapping quality alignments are likely to have originated from the same long template as the ambiguous alignment and can serve as “links” to guide the ambiguously mapping read to its correct location in the genome. Different flow cell and genomic distance thresholds can be evaluated simultaneously to identify links for these ambiguously mapped reads. In some embodiments, any sequence read with more than one alignment may be considered ambiguous. Accordingly, by way of a non-limiting example, at step 220, the method may proceed by identifying a polynucleotide sequence read with an alignment field indicating that the first polynucleotide sequence read ambiguously. That alignment field may be the alignment field that contains the alignments to two or more locations in the target polynucleotide sequence.

Genomic information may also be obtained directly from a sequencing system. The sequencing system may generate raw data in the form of nucleic acid sequence reads, and the corresponding pixel, intensity, or location on a substrate where that sequence read was sequenced. These reads can then be processed using alignment methods to map them to a reference genome, identify variations, and reconstruct genomic sequences. Raw data obtained from a sequencing system may include processing to convert the data into information on the sequence of the polynucleotides bound to the flow cell. This may involve intermediary steps such as quality control, removing adapter sequences, and trimming low-quality bases. In some embodiments, alignment processes may be applied before or after such steps and may be iteratively applied to map the reads to a reference genome. In some embodiments, the system may map the reads, allowing for downstream analyses such as variant calling or structural variant identification.

The data obtained from spatially linked read pairs may be distinct from that of, for example, barcoded read pairs due to the way information is captured and utilized. Spatially linked read pairs may involve associating the physical positions of DNA sequences on a sequencing substrate. This means that the data provides insights into the two-dimensional placement of genetic material on a sequencing substrate. This information can be valuable for understanding whether different read pairs came from a single sequence. On the other hand, barcoding read pairs typically involves adding short DNA sequences (barcodes) to the DNA fragments before sequencing. These barcodes serve as molecular “tags” that help distinguish and track different DNA fragments from the same source. The primary purpose of barcoding is often to associate related reads, ensuring they come from the same genomic template. Source information and proximity information for read pairs relate to the relationship between two reads, but they focus on different aspects.

Source information refers to the origin or source of the two reads within a read pair. In other words, it indicates which template nucleic acid or genomic region the two reads were derived from. This information may be used to correctly associate reads that are part of the same genomic fragment or template. Source information is typically obtained through barcoding or other labeling methods. For example, each DNA fragment might be assigned a unique barcode before sequencing, so when two reads share the same barcode, it means they come from the same original DNA template.

Proximity information, on the other hand, relates to the physical distance between the two reads within a read pair. This information is particularly relevant when reads are generated from spatially arranged templates, such as in spatial transcriptomics or spatial genomics. Proximity information indicates that the two reads were captured from nearby physical locations on a substrate or within a tissue. This information provides insights into the spatial relationships and organization of genetic material, revealing how different genomic elements are positioned relative to each other. While both source and proximity information may be associated with read pairs, they may serve different purposes. Source information helps correctly link reads that belong to the same template, while proximity information provides insights into the local connectivity of read pairs. In some embodiments, these two types of information might be used together to better identify structural variants.

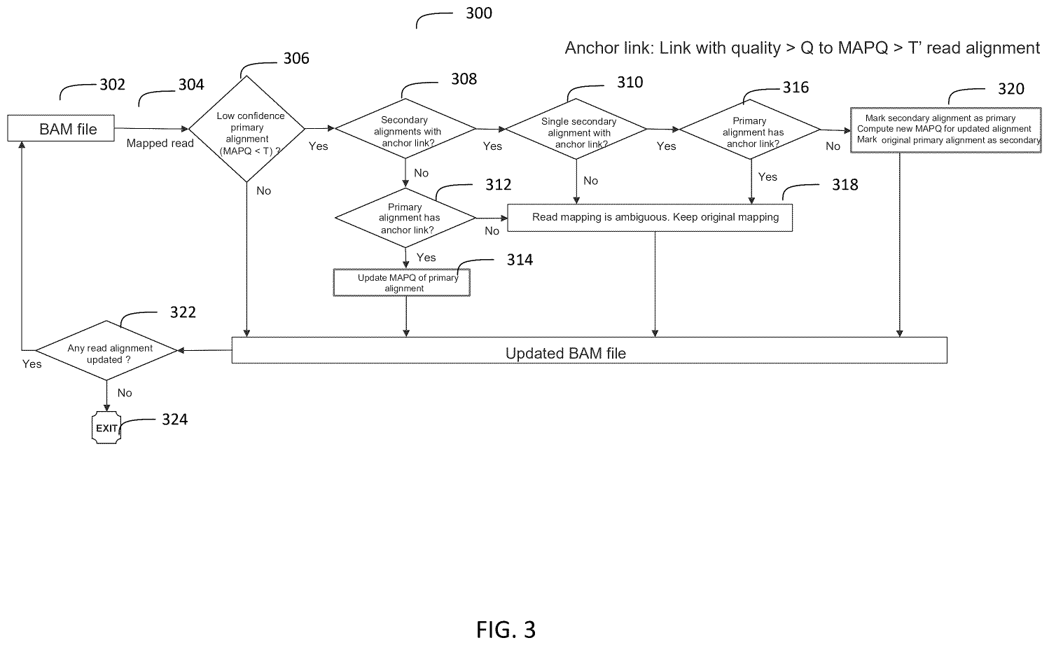

FIG. 3 presents an iterative method 300 designed to refine alignments in sequencing data by harnessing spatial information in order to update a BAM file. This method commences with the processing of a BAM file 302, which may include a plurality of mapped sequence reads with various levels of confidence and unmapped reads. The BAM file 302 is a binary version of a SAM file and is used in bioinformatics for storing sequence data, especially alignments of reads to a reference genome. The BAM file is commonly used in the bioinformatics field as a compressed, binary format that allows for efficient storage and quick random access of data, making it suitable for large-scale genomics projects.

From this BAM file 302, mapped reads 304 are selected. These mapped reads 304 are the results from aligning sequences reads to a reference genome. Some of these mapped reads 304 will be accurately mapped, some may be ambiguous (having multiple potential mapping locations), and some may not be mapped at all. Initially, each mapped read's primary alignment undergoes an evaluation for its confidence level determined by its MAPQ value at a decision step 306. During the decision 306, the process 300 evaluates the confidence of these mapped reads using by referring to the mapping quality (MAPQ) of each mapped read. The MAPQ gives an estimate of the probability that a mapped read's alignment is incorrect. If, at the decision step 306, a read has a MAPQ score less than a threshold T, it is considered to have a low-confidence primary alignment and the process 300 moves to a decision step 308 to determine if the mapped read has a secondary association with an anchor link. Alignments verified to have an anchor link may have their MAPQ updated according to whether the linked read increases the confidence of the mapping. If the confidence of the mapped read is greater than the threshold T at the decision step 306, then the process 300 moves to update the BAM file.

If the process 300 moved to decision step 308 to determine if there are secondary alignments with an anchor link, a determination is made whether an anchor link can provide additional spatial context, and help to refine where the ambiguous sequence read might best align to the reference genome. If there is no secondary alignment with an anchor link, the process 300 moves to a decision step 312 to determine if the primary alignment has an anchor link. If the primary alignment has an anchor link, the MAPQ score of the ambiguous sequence read is updated to reflect this mapping information from the anchor link.

If the secondary alignment has an anchor link at decision step 308, then the process 300 moves to a decision step 310 to determine if there is a single, as opposed to multiple, secondary alignments that features an anchor link. If there are single, secondary alignments with an anchor link at the decision step 310, then the process 300 moves to a decision step 316, where the process 300 may check whether the primary alignment has an anchor link. If, at decision step 316, the primary alignment has no anchor link, but the secondary alignment possesses an anchor link as determined earlier, the mappings between the primary and secondary alignments may be swapped at a step 320. The secondary alignment may assume primary status, and the MAPQ may be recalculated for this newly designated alignment, and the initial primary alignment may be relegated to secondary status. Conversely, if both the primary and secondary alignments exhibit anchor links (see decision steps 308 and 312) or the primary alignment has a link does regardless of if the secondary alignment has a link (see decision steps 310 and 316), the system recognizes the read mapping as ambiguous at step 318, and in such instances, the original mapping may be preserved without any alterations.

The final phase of the method may involve a check, at decision step 322, across all reads to ascertain if any of their alignments underwent updates during this iteration. In cases where no updates are identified, the process may finish at step 324. However, if updates are detected, the cycle may continue and repeat by updating the BAM file 302 with the revised information as a new starting point for the process 300. The goal of this procedure is to increase the accuracy of mapped reads by using spatial information, with a specific emphasis on anchor links, to enhance the alignments in the BAM file.

Some applications of the disclosure may employ a method to improve the confidence in the mapping quality of sequence alignments, particularly in cases where reads are mapped to multiple locations in a genome. As described herein, the MAPQ score serves as an indicator of the degree of confidence in an alignment's accuracy. MAPQ is a statistical measure that indicates the probability that a read is correctly mapped to a particular location in the genome. A MAPQ score of 0 indicates no confidence, while higher scores indicate increasing confidence.

According to the methods described herein, the MAPQ score of a multi-mapped read may be updated by using spatial location information from anchor reads. In some embodiments, anchor reads may be reads with a high MAPQ score (>30) that map uniquely to the genome. These high-confidence reads may be used as described above, to determine the correct location of multi-mapped reads (reads with a MAPQ of 0), at which point the newly mapped read may be given an improved MAPQ value. For instance, if a multi-mapped read is flanked by anchor reads that are within a certain distance threshold (50 kb in the example given) and/or are linked with a sufficient link quality score, then the MAPQ score of the multi-mapped read is updated to reflect a higher confidence in its placement. Such methods may involve analyzing multiple top-scoring candidate alignments for each read. The method can be implemented directly within a mapper as multiple candidate alignment locations are scored and compared or as an alignment post-processing step wherein the top-scoring alignments can be obtained by programming existing mappers to dump out secondary alignments (up to a threshold alignment score worse than the best scoring primary alignment).

While the method above describes disambiguation of mapping quality zero read alignments, the same method is applied to also correct potentially mismapped alignments that have been incorrectly assigned a moderate to high mapping quality (e.g., mapping quality less than 30). Various methods are available for finding anchor reads. Anchor reads may be determined beforehand, one at a time, and in batches.

The confidence score for an alignment, often represented as a MAPQ score in sequencing, can be updated through various methods to ensure higher accuracy in read placement. These methods generally involve the utilization of nearby high-confidence reads, referred to herein as anchor reads, and the spatial and alignment score relationships between these reads and the multi-mapped reads in question. The disclosure contemplates several approaches to update the confidence score of an alignment. For example, one method of updating the alignment score may rely on copying the alignment score of anchor reads. In some cases, the confidence score of a multi-mapped read could be directly influenced by the alignment scores of nearby anchor reads. If an anchor read has a high alignment score and is spatially linked to the multi-mapped read such that the read's placement is unambiguously resolved, the alignment score of the multi-mapped read might be adjusted to mirror the high confidence of the anchor read.

Other methods may involve a new evaluation of the confidence of the link. For example this method may involve evaluating the strength of the link between the multi-mapped read and its potential alignments, as well as the link between the multi-mapped read and other nearby high MAPQ anchor reads. A stronger link quality may lead to a higher confidence score for the multi-mapped read. A composite method may also be used where both the spatial links to anchor reads and the alignment scores are taken into account. For example, a link quality score, the MAPQ of the anchor reads, and the alignment scores of alternative locations can be combined into a composite score that updates the MAPQ for a multi-mapped read. The MAPQ score may also be updated if the alignment meets certain threshold criteria. For instance, if only one alternative alignment is within a certain distance from the high MAPQ anchor reads and the alignment score is above a set threshold, then the confidence score of the multi-mapped read may be increased by a percentage or multiplicative factor. As another example, after the initial alignments are generated, a post-processing step can reassess the MAPQ scores by considering additional information, such as the distribution of high MAPQ reads across the genome or the sequencing depth in different regions.

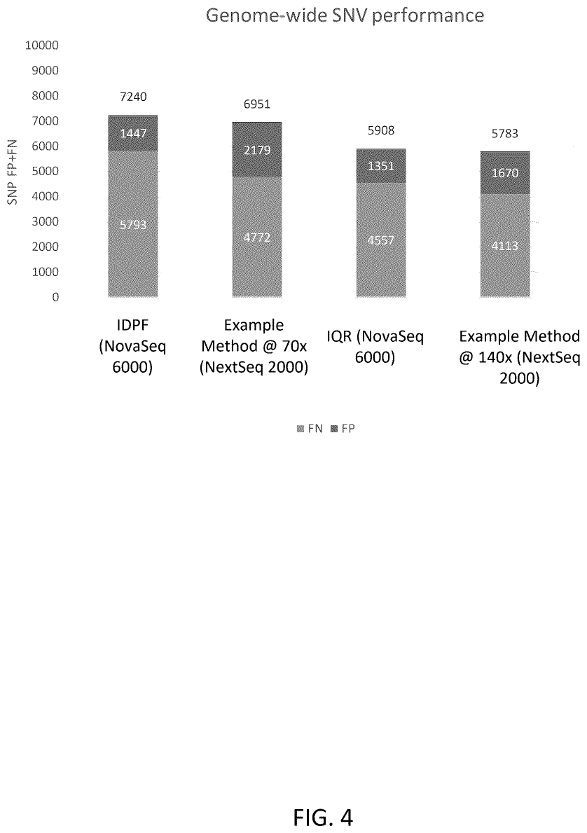

FIG. 4 is a histogram that displays the performance of various sequencing technologies and methods in detecting single nucleotide variants (SNVs) across the genome. The Y-axis represents the count of SNPs that are either false negatives (FN) or false positives (FP). The X-axis displays different sequencing technologies or methods.

The chart demonstrates that the performance of the disclosed methods, especially with loading samples at 70× coverage, have the capacity to reduce false negatives. While these results show a minor increase in false positives, further improvement is expected as higher link rates are established. The data shown is of MAPQ of 25 or more with a linking rate of 32%, and the updated alignments used anchor reads with MAPQ≤30. This will potentially enhance the disclosed methods' performance, even at lower coverage levels. The chart specifically shows that the example method @70× (NextSeq 2000) demonstrates the best performance in terms of false negatives (FN), with the lowest count among the technologies/methods presented. In contrast, IDPF (NovaSeq 6000) has the highest count of false negatives. Both versions of the example methods (70× and 140×) have a comparable count of false positives, but the 140× version has notably fewer false negatives than its 70× counterpart. In general, increasing coverage should help reduce FNs as these reads can contribute as variant evidence.

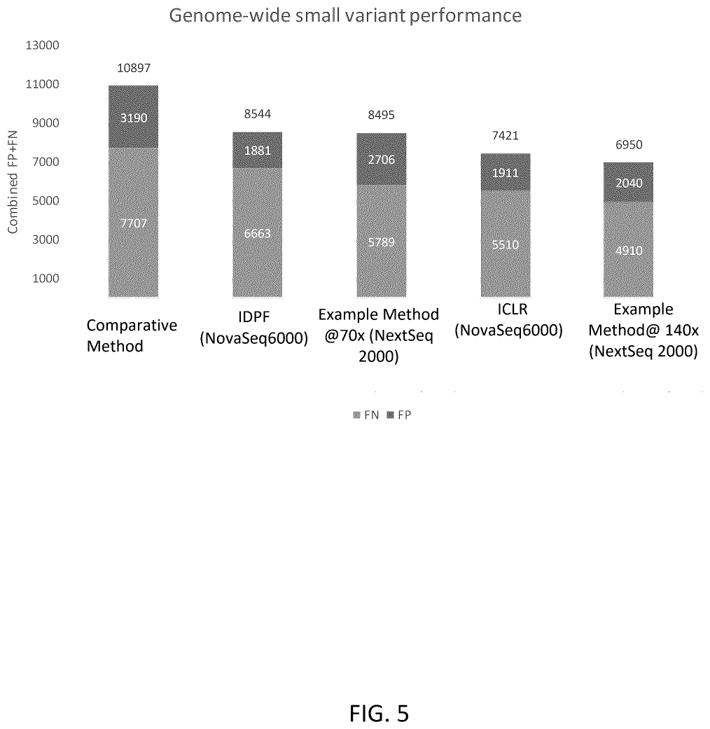

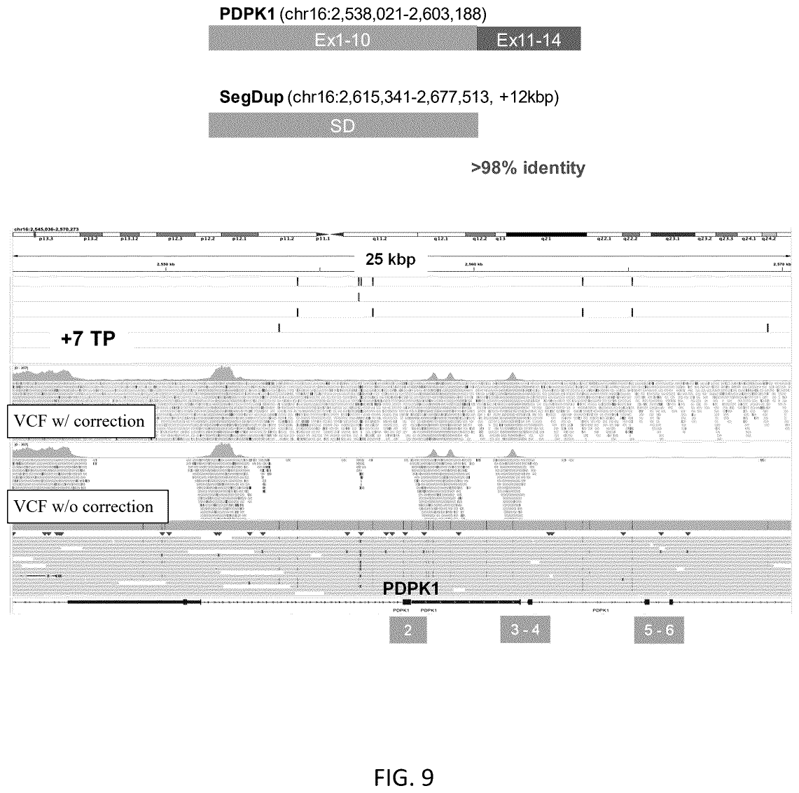

FIG. 5 presents a bar chart illustrating the genome-wide small variant performance across different sequencing technologies: a comparative method, IDPF, ICLR, and two versions of example methods according to the disclosure (70× and 140× on NextSeq 2000). The vertical axis quantifies combined false negatives (FNs) and false positives (FPs). Notably, the example methods, especially at 140× (NextSeq 2000), display a considerable decrease in FNs compared to its counterparts. Thus, consistent with the disclosure, additional methods may be used to identify and reduce these false positives. In some embodiments, a false positive detection method may be used that is adapted for false positive types and amounts that are present in sequencing data that includes physical location data and/or link data between reads. In FIG. 5, the accompanying data specifies that the displayed results for the example methods are based on a quality score of 25, with a 32% linking rate, and that reads with a MAPQ value of 30 or below have been corrected. Furthermore, FIG. 9 underscores the advantage of certain example methods in bypassing PCR-induced errors evident in ICLR and avoiding the systematic biases inherent in traditional long-read sequencing. This distinction highlights the disclosed method's accuracy and efficiency in determining genome-wide small variants.

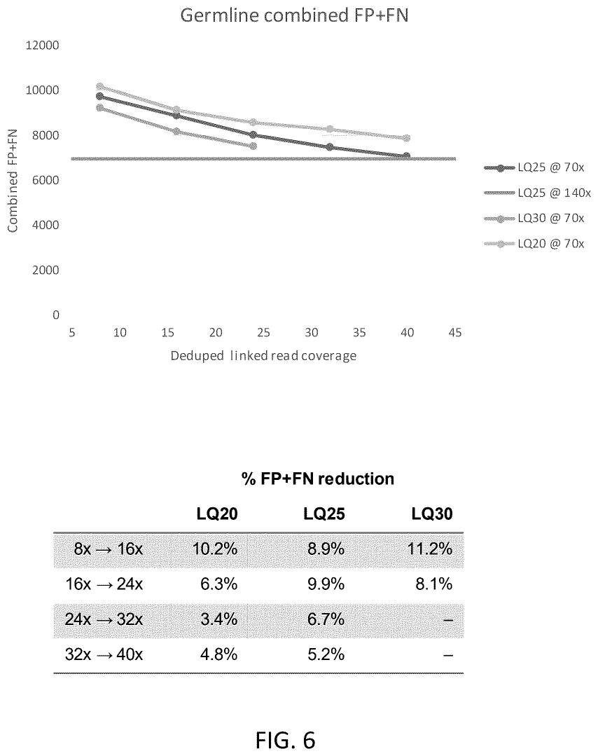

FIG. 6 similarly showcases the relationship between deduplicated linked read coverage and combined false positives and negatives (FP+FN) for germline sequencing. The graph depicts four distinct curves representing different link quality scores: LQ25 at 70×, LQ25 at 140×, LQ30 at 70×, and LQ20 at 70×. As the deduplicated linked read coverage increases, there is a decline in the combined FP+FN across all quality scores. Notably, the gains from higher link rates start to show diminishing returns around the 32× coverage mark. The table supplementing the graph further details the percentage improvements in performance between different coverage intervals for the LQ20, LQ25, and LQ30 scores. One potential strategy for the example methods would be targeting Q25 links with a link rate between 45% to 50% at 70× coverage, ensuring a coverage of linked data of 30× or more. This strategy is likely to yield an effective performance in terms of minimizing errors.

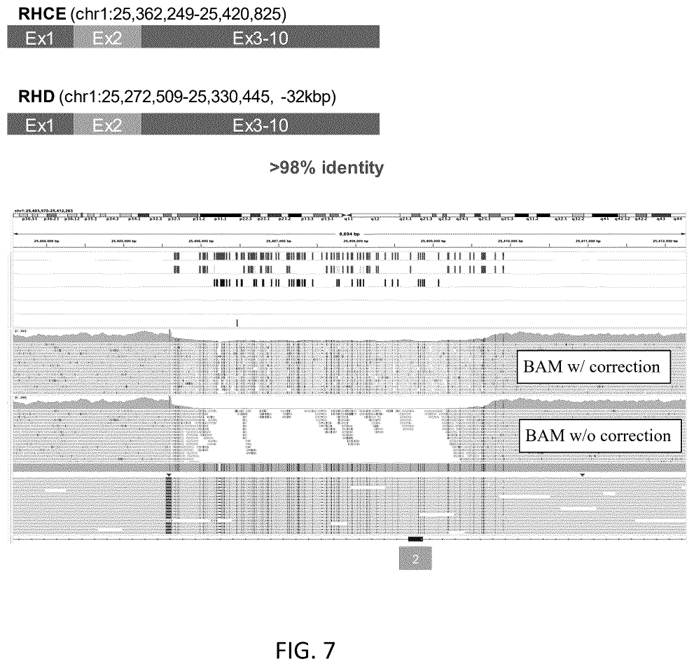

FIG. 7 includes two panels and presents a visualization focusing on the RHCE gene, which encodes the Rh blood group, commonly known as Rh positive or Rh negative. In the first panel, adjacent and below the RHCE gene is the RHD gene, another gene for the Rh blood group. Notably, RHD shares a 98% identity with RHCE, with specific regions such as exon 2 exhibiting such high similarity that it poses challenges in accurate mapping. This close resemblance between the two genes can lead to gene conversions, subsequently causing variations in the Rh blood group.

The relevant region in the panels spans an 8.5 kbp segment. Every sequenced read with a MAPQ score greater than 0 is showcased. Among the displayed reads, those highlighted in red have been corrected using spatial information by the disclosed methods, which demonstrates that this method of correction enhances the accuracy and reliability of the sequencing data. The experiment was performed using the CMRG v1.0 truthset. The sequencing data is from NextSeq 2000, with a coverage of approximately 140×. Notably this approach recovered 63 out of 64 false negatives, which significantly bolsters the accuracy of the data.

The lower panel provides a more detailed view of the sequencing data. The top region of the stacked chart shows a linear representation of base pairs within a range, with the central portion of the figure highlighting an 8.5 kbp segment. The subsequent layers offer insights into various sequencing details. The top two tracks, correspond to “corrected” and “uncorrected,” represent the reads from the example sequencing platform. The color distinction, especially the red reads in the “corrected” track, indicates sequence reads that have undergone correction using spatial information.

Three distinct variant file tracks follow, highlighting locations of single nucleotide polymorphisms (SNPs) or other genetic variations. The main body of the figure is populated by horizontal lines or reads, with various color codes. These represent sequences mapped at specific positions. Two sections are particularly highlighted: “BAM w/correction” and “BAM w/o correction.” These sections showcase the alignment of reads from the example sequencing platform, both before and after corrections were applied. The gray-filled areas underneath these tracks represent coverage, or the number of reads that align to that particular position.

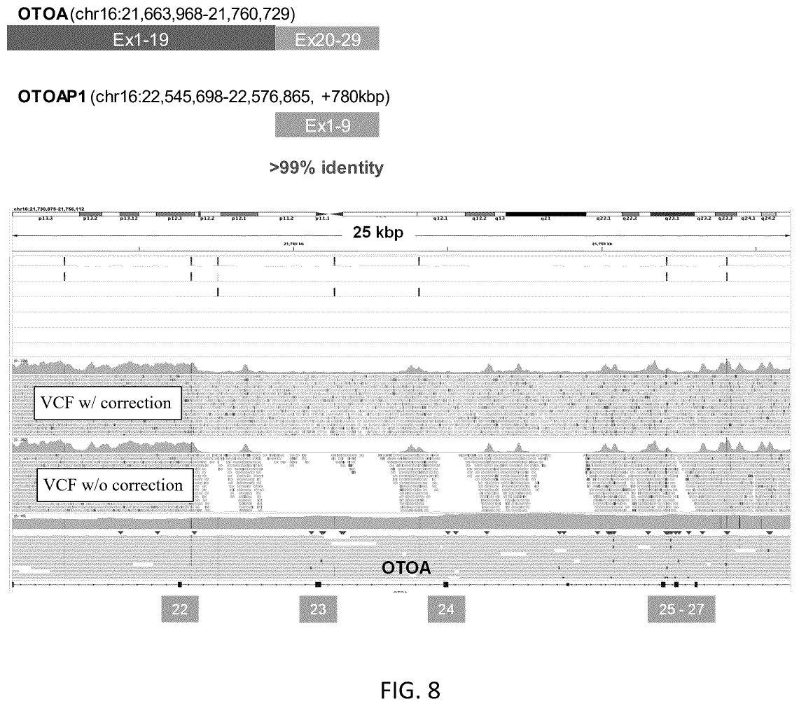

FIG. 8 relates to the performance of the disclosed methods towards OTOA mutations that are associated with AR non-syndromic deafness. The pseudogene OTOA1, 780 kbp upstream, has high identity with OTOA ex 20-29. The first panel includes a cartoon pictorially demonstrating the structural similarities, and includes regions labeled “Ex1-19” and “Ex20-29”. Below this, there's a smaller gene segment labeled “Ex1-9”, which is highlighted to have >99% identity with a portion of the gene segment above it. This high percentage of identity between segments can pose challenges in traditional sequencing, as distinguishing between such similar sequences can be difficult. However, the disclosed method demonstrates that 3/3 false negatives recovered, and 0 false positives added when analyzing this gene.

The panel below includes a detailed sequencing view of a 25 kbp genomic region. Multiple tracks are layered to showcase different types of sequencing reads and their corresponding alignments. The top two tracks “VCF w/correction” and “VCF w/o correction”-highlight the difference in sequencing reads when using an example correction feature. “Recovered GIAB v4.2.1 FNs”—This track emphasizes the false negatives that have been recovered using the example sequencing platform. “Remaining FNs”—Shows the false negatives that remain even after correction. “FPs”—Refers to the false positives identified in the sequencing data. Below these overview tracks are more detailed tracks depicting sequencing coverage. Again, there is distinction between “corrected” and “uncorrected” coverage, with the corrected views being more comprehensive. Each horizontal line within these coverage regions represents individual sequencing reads. The gray-filled areas underneath these tracks represent coverage, or the number of reads that align to that particular position, and the red reads indicate recovered reads.

FIG. 9 is a visualization depicting the sequencing challenges surrounding the PDPK1 gene and the results of using an embodiment of the disclosed methods. The PDPK1 gene is implicated in specific types of cancer, including prostate and non-small cell lung cancer. The presence of a segmental duplication near this gene presents significant sequencing challenges, primarily due to the high similarity (98% identity) between the PDPK1 gene (specifically exons 1-10) and this duplication.

In the visualization there are two bar representations. The upper bar depicts the PDPK1 gene structure, split into two sections—“Ex1-10” and “Ex11-14”, indicating different exon regions. Below this, there is a longer bar labeled “SD” (Segmental Duplication) which is nearly identical (>98%) to the “Ex1-10” section of PDPK1, and the structural similarity is highlighted by the matched colors/shading between the two corresponding sections. The presence of such segmental duplications in genomes often complicates sequencing because reads from one region can be mistakenly mapped to the other, especially if the two regions are nearly identical.

The lower panel includes more detailed sequencing data centered on sequencing results in the vicinity of the PDPK1 gene. Here multiple tracks are stacked vertically. These tracks display how sequencing reads are mapped to the reference genome. The top stacks VCF w/correction and VCF w/o correction illustrate the mapping of sequencing reads using the example sequencing platform, both with and without a correction feature. The difference in read mapping between these conditions can be observed. The main body of the figure is populated by horizontal lines as reads, with grey lines indicating the originally aligned reads, and the red reads showing the reads rescued by the disclosed methods. The stacked grey lines underneath these tracks represent coverage, or the number of reads that align to that particular position.

A noteworthy point highlighted in the top left corner is the successful recovery of 7 out of 8 false negatives without introducing any false positives. This achievement underscores the efficacy of the sequencing method employed, especially given the challenges presented by the proximity of the segmental duplication.

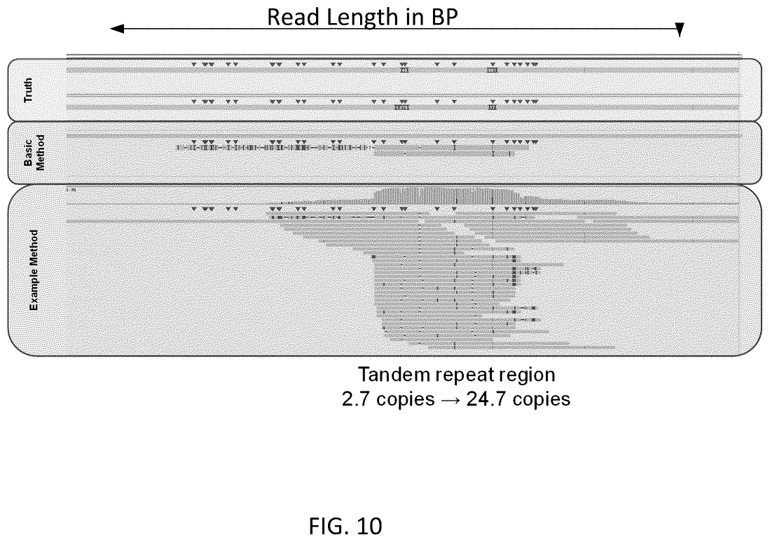

FIG. 10 compares the performance of two sequencing assembly technologies, a standard assembly method and the example methods, in detecting deletions (represented as “DEL1” and “DEL2”) in a reference genome. The topmost Panel (ref.fasta) represents the reference genome sequence, serving as the baseline for comparisons. The two rows HG002_hap1.bam and HG002_hap2.bam represent the true haplotypes or variations present in the sample. The red blocks, labeled as “DEL1” and “DEL2”, denote two deletions present in the actual data. The rows related to the comparative assembly system consists of two rows, which represent assemblies generated by the system. The first shows contigs, or assembled sequences, produced by the comparative system. The second shows the contigs are extended into scaffolds, which are longer sequences that may include gaps (often filled with unknown bases). This row shows that standard assembly detected “DEL1” accurately but missed “DEL2”.

In comparison, the rows related to the example methods detected both “DEL1” and “DEL2”, which suggests a more comprehensive detection capability for these specific deletions. However, the fragmented contigs in the “contigs.bam” row might indicate that the assembly is less continuous or more broken up compared to the standard assembly method.

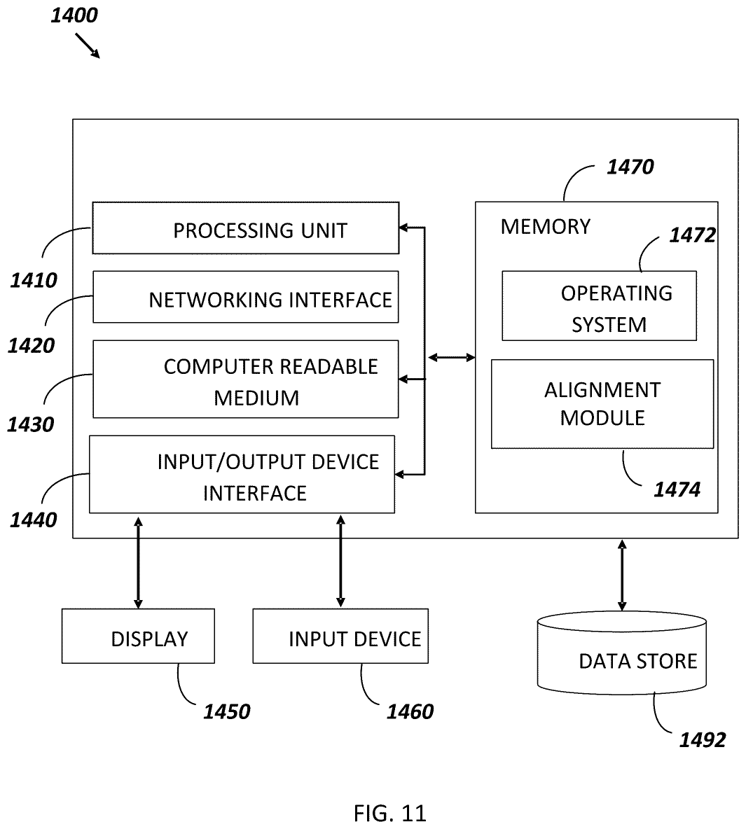

Embodiments of the present disclosure also include a system for analyzing and assembling sequences of polynucleotides. FIG. 11 is a diagram of an exemplary computing system 1400 that may be used in connection with an illustrative sequencing system. The computing system 1400 may be configured to determine a DNA sequence by using the sequencing and assembly methods disclosed herein. The general architecture of the computing system 1400 includes an arrangement of computer hardware and software components. The computing system 1400 may include many more (or fewer) elements. It is not necessary, however, that all of these generally conventional elements be shown in order to provide an enabling disclosure.

As illustrated, the computing system 1400 includes a processing unit 1410, a network interface 1420, a computer-readable medium drive 1430, an input/output device interface 1440, a display 1450, and an input device 1460, all of which may communicate with one another by way of a communication bus. The network interface 1470 may provide connectivity to one or more networks or computing systems. The processing unit 1410 may thus receive information and instructions from other computing systems or services via a network. The processing unit 1410 may also communicate to and from memory 1470 and further provide output information for an optional display 1450 via the input/output device interface 1440. The input/output device interface 1440 may also accept input from the optional input device 1460, such as a keyboard, mouse, digital pen, microphone, touch screen, gesture recognition system, voice recognition system, gamepad, accelerometer, gyroscope, or other input device.

The memory 1470 may contain computer program instructions (grouped as modules or components in some embodiments) that the processing unit 1410 executes in order to implement one or more embodiments. The memory 1470 generally includes RAM, ROM and/or other persistent, auxiliary or non-transitory computer-readable media. The memory 1470 may store an operating system 1472 that provides computer program instructions for use by the processing unit 1410 in the general administration and operation of the computing device 1400. The memory 1470 may further include computer program instructions and other information for implementing aspects of the present disclosure.

For example, in one embodiment, the memory 1470 includes an alignment module 1474 for analyzing and assembling sequences of polynucleotides. The module 1474 can perform the methods disclosed herein, including the method described with respect to the flow diagrams of, for example, FIG. 2. In addition, memory 1470 may include or communicate with the data store 1490 and/or one or more other data stores that store one or more inputs, one or more outputs, and/or one or more results (including intermediate results) of determining a DNA sequence and providing an assembly process according to the present disclosure.

### Systems and Instruments

An aspect of the disclosure is directed to methods for identifying links across an entire genome. In some embodiments, a method may include receiving a BAM file that includes spatial information. The method may proceed by splitting the BAM file into surface and chromosomes. For each subset of the BAM file (surface/chromosome), a “KD-tree” may be constructed, which is a data structure for querying m-dimensional ranges, where m>1. Then, the method may proceed for each point p in each KD-tree t. The KD-tree t may in turn be queried for all points p_neighbors within spatial distance threshold of p. The KD-tree t may be queried for each p2 in p_neighbors. The method may determine a link if p and p2 are within a genomic distance threshold, and then record (p,p2) as a link.

In some embodiments, a method of finding links between read pairs on a flow cell, may include the step of providing sequencing data for read pairs from clusters on the flow cell. The method may also include filtering clusters that are spatially distant from one another, and/or filtering clusters that are genomically distant from one another. The method may include selecting neighboring clusters that are within a spatial distance threshold as neighboring clusters; and the assigning links to two read pairs in the neighboring clusters when the clusters are within a genomic distance threshold. In some embodiments, assigning links to two read pairs may occur when the genomic distance threshold is a preset threshold. In some embodiments, the method may generate a first subset of the sequencing data for read pairs by selecting the clusters that are spatially distant from one another. In some embodiments, the method may include selecting a first cluster that has nucleic acid derived from a first chromosome and a second cluster has nucleic acid from a second, different chromosome.

In some embodiments, the clusters are on the same surface but opposite ends of the flow cell. In some embodiments, the clusters are opposite surfaces of the flow cell. In some embodiments, the calculating the spatial null distribution of the plurality of the plurality of read pairs comprises determining read pairs from clusters on the flow cell. In some embodiments, a first cluster has nucleic acid derived from a first chromosome and a second cluster has nucleic acid from a second, different chromosome.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or step diagrams of methods, apparatus (systems), and computer program products according to embodiments of the disclosure. It will be understood that each step of the flowchart illustrations and/or step diagrams, and combinations of steps in the flowchart illustrations and/or step diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or step diagram step or steps. These computer readable program instructions may also be stored in a computer readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart(s) and/or step diagram(s) step or steps.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or step diagram step or steps. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and step diagrams in the Figures illustrate the architecture, functionality, and operation of possible implementations of systems, methods, and computer program products according to various embodiments of the present disclosure. In this regard, each step in the flowchart or step diagrams may represent a service, module, segment, or portion of instructions, which comprises one or more executable instructions for implementing the specified logical function(s). In some alternative implementations, the functions noted in the steps may occur out of the order noted in the Figures. For example, two steps shown in succession may, in fact, be executed substantially concurrently, or the steps may sometimes be executed in the reverse order, depending upon the functionality involved. In addition, certain steps may be omitted in some implementations. The methods and processes described herein are also not limited to any particular sequence, and the steps or states relating thereto can be performed in other sequences that are appropriate.

It will also be noted that each step of the step diagrams and/or flowchart illustration, and combinations of steps in the step diagrams and/or flowchart illustration, can be implemented by special purpose hardware-based systems that perform the specified functions or acts or carry out combinations of special purpose hardware and computer instructions. For example, any of the processes, methods, algorithms, elements, steps, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

In some embodiments, the methods may be written in any of various suitable programming languages, for example compiled languages such as C, C#, C++, Fortran, and Java. Other programming languages could be script languages, such as Perl, MATLAB, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (for example, iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is commonly via Internet connection, either wireless or by a network cable through an access point. In some embodiments, a storage device may be maintained and managed by the individual or entity directly associated with an assay instrument, whereas in other embodiments a storage device may be maintained and managed by a third party, commonly at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations. One aspect of the disclosure is directed to a workflow module that may be integrated into existing workflows. In some embodiments, a workflow module may be a two-channel sequencing module and may be integrated into a NGS sequence analysis platform, for example the DRAGEN™ Bio-ID platform from Illumina.

### Definitions

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “polynucleotide,” “oligonucleotide,” “nucleic acid” and “nucleic acid molecules” are used interchangeably herein and refer to a covalently linked sequence of nucleotides of any length (i.e., ribonucleotides for RNA, deoxyribonucleotides for DNA, analogs thereof, or mixtures thereof) in which the 3′ position of the pentose of one nucleotide is joined by a phosphodiester group to the 5′ position of the pentose of the next. The terms should be understood to include, as equivalents, analogs of either DNA, RNA, cDNA, or antibody-oligo conjugates made from nucleotide analogs and to be applicable to single stranded (such as sense or antisense) and double stranded polynucleotides. The term as used herein also encompasses cDNA, which is complementary or copy DNA produced from an RNA template, for example by the action of reverse transcriptase. This term refers only to the primary structure of the molecule. Thus, the term includes, without limitation, triple-, double- and single-stranded deoxyribonucleic acid (“DNA”), as well as triple-, double- and single-stranded ribonucleic acid (“RNA”). The nucleotides include sequences of any form of nucleic acid. As apparent from the examples below and elsewhere herein, a nucleic acid can have a naturally occurring nucleic acid structure or a non-naturally occurring nucleic acid analog structure. A nucleic acid can contain phosphodiester bonds; however, in some embodiments, nucleic acids may have other types of backbones, comprising, for example, phosphoramide, phosphorothioate, phosphorodithioate, O-methylphosphoroamidite and peptide nucleic acid backbones and linkages. Nucleic acids can have positive backbones; non-ionic backbones, and non-ribose based backbones. Nucleic acids may also contain one or more carbocyclic sugars. The nucleic acids used in methods or compositions herein may be single stranded or, alternatively double stranded, as specified. In some embodiments a nucleic acid can contain portions of both double stranded and single stranded sequence, for example, as demonstrated by forked adapters. A nucleic acid can contain any combination of deoxyribo- and ribonucleotides, and any combination of bases, including uracil, adenine, thymine, cytosine, guanine, inosine, xanthanine, hypoxanthanine, isocytosine, isoguanine, and base analogs such as nitropyrrole (including 3-nitropyrrole) and nitroindole (including 5-nitroindole), etc. In some embodiments, a nucleic acid can include at least one promiscuous base. A promiscuous base can base-pair with more than one different type of base and can be useful, for example, when included in oligonucleotide primers or inserts that are used for random hybridization in complex nucleic acid samples such as genomic DNA samples. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole and 3-nitropyrrole. Promiscuous bases that can base-pair with at least two, three, four or more types of bases can be used.

As used herein, phrases and/or terms related to probabilities refer to the probability of the fragments of the polynucleotide being located near each other on the flow cell. Relevantly, the probability may be correlated with a distance between the fragments of the polynucleotide in the polynucleotide. In the context of sequencing technologies like those used in next-generation sequencing (NGS), a polynucleotide is a long chain of nucleotides, which can be DNA or RNA. The process typically involves fragmenting these long molecules into smaller pieces for easier sequencing. When these fragments are placed onto a flow cell, which is a glass slide used as a solid surface for sequencing reactions, they are typically spatially distributed in an even and random distribution across the flow cell surface.

The probability that fragments of a polynucleotide are located near each other on the flow cell being correlated with the distance between the fragments in the actual polynucleotide chain refers to the likelihood that segments that are closer together along the chain of the polynucleotide will end up being positioned closer to each other on the flow cell during sequencing. This correlation may be a result of the fragmentation process, or the method used to immobilize the fragments onto the flow cell.

For example, if the fragmentation method merely cuts the polynucleotide randomly and then casting the fragments onto the flow cell, there is a chance that fragments from the same region of the original polynucleotide will be in proximity on the flow cell. However, if the fragments are attached to the flow cell in a random fashion, then the initial proximity in the polynucleotide may not be preserved. On the other hand, certain methods, such as those according to the disclosure, may retain some spatial information, leading to a higher probability that closely located sequences in the polynucleotide remain close on the flow cell. The relevance of this correlation depends on the sequencing technology used. In some technologies, preserving the relative positions of the fragments can be beneficial for reconstructing the original sequence of the polynucleotide, as it can aid in the mapping and assembly process. Here, “Spatially co-located” in the context of genomic fragments refers to the occurrence of two or more DNA fragments being positioned in close physical proximity to one another in a given space. This term is used to describe how fragments are situated relative to each other on the flow cell during sequencing.

As used herein, the term “fragment,” when used in reference to a first nucleic acid, is intended to mean a second nucleic acid having a part or portion of the sequence of the first nucleic acid. Generally, the fragment and the first nucleic acid are separate molecules. The fragment can be derived, for example, by physical removal from the larger nucleic acid, by replication or amplification of a region of the larger nucleic acid, by degradation of other portions of the larger nucleic acid, a combination thereof or the like. The term can be used analogously to describe sequence data or other representations of nucleic acids. As used herein, the term “haplotype” refers to a set of alleles at more than one locus inherited by an individual from one of its parents. A haplotype can include two or more loci from all or part of a chromosome. Alleles include, for example, single nucleotide polymorphisms (SNPs), short tandem repeats (STRs), gene sequences, chromosomal insertions, chromosomal deletions etc. The term “phased alleles” refers to the distribution of the particular alleles from a particular chromosome, or portion thereof. Accordingly, the “phase” of two alleles can refer to a characterization or representation of the relative location of two or more alleles on one or more chromosomes.

As used herein, the term “active region” or “region of interest” refers to a segment of the genome that is specifically targeted for sequencing or currently being analyzed during a sequencing method step. These regions may be a single region or a window covering multiple sequence reads at a time. When it comes to methods of assembly or structural variant detection, an active region is often the focal point where advanced sequencing techniques are applied to obtain a highly accurate sequence. In the context of structural variant detection, active regions may be scrutinized using specialized techniques that can detect larger-scale genomic alterations, such as inversions, translocations, or large indels. These variants may not be evident with standard sequencing approaches and often require methods like paired-end or long-read sequencing to span the entire region of interest. This is also relevant for assembling a genome from scratch, where active regions may be targeted for individual steps of a sequencing process to be sequenced with a higher coverage depth or with longer reads to ensure that these important parts of the genome are assembled correctly.

As used herein, the term “Anchor Read” refers to reads that can be mapped with high confidence or unambiguously to unique positions in a genome. Anchor reads serve as reliable reference points in the mapping process, providing high-confidence alignments between the sequence reads and the reference genome. These anchor reads are usually characterized by a high degree of similarity to known sequences in the reference genome, often facilitated by processes that assign high-quality alignment scores based on the number of matches, mismatches, gaps, and other criteria.

As used herein, the term “flanking” genomic sequencing refers to stretches of DNA or RNA fragments that are situated at a certain distance from a specific region of interest, such as an anchor read, a gene, a mutation site, or a repetitive element. These regions may be used as reference points and may not necessarily be directly next to the region of interest. The distance between the flanking region and the target can vary widely, from just a few base pairs to several kilobases away, depending on the genome and the method of used to link reads to anchor reads. For example, some methods of the disclosure are able to link reads from several kilobases away, and may be even more sensitive to structural variants that are several kilobases long.

In the context of anchor sequence reads, as described above, flanking regions serve as reference points for alignment but are not required to be immediately adjacent to the sequence of interest. An anchor read may include sequences that are several hundred or even thousands of base pairs away from the flanking regions. These non-adjacent flanking regions are particularly useful when the anchor read includes repetitive sequences that occur frequently in the genome, or in identifying structural variants. By identifying unique flanking sequences at a distance, methods according to the disclosure can still map the anchor read to the correct location on the genome.

The use of distant flanking regions is a useful strategy of the disclosure for use in genomic sequencing to achieve accurate mapping. It allows for the unambiguous alignment of reads that would otherwise be difficult to place due to the presence of repetitive or complex sequences. By considering a range of distances for potential flanking regions, various tools can effectively ‘anchor’ reads to their proper location in the genome, which is useful for reliable genome assembly and the accurate identification of genetic variants.

As used herein, the term “unambiguous mapping,” in the context of genomic sequencing refers to the process of correctly and uniquely assigning a sequenced DNA fragment to a single location in a reference genome. This means that the sequence of the fragment is so distinctive that it matches one and only one region in the reference genome with a high degree of confidence. By way of example, challenges in mapping may arise because genomes often contain repetitive sequences. If a fragment comes from a repetitive region, it may map to multiple locations, leading to ambiguous mapping. Ambiguity in mapping can complicate genetic analyses and may lead to incorrect conclusions. Therefore, the goal is to achieve unambiguous mapping wherever possible, which is more likely with longer reads, longer synthetic reads, long sequences of linked reads, or with fragments that include unique sequences flanking repetitive regions.

As used herein, the term “ambiguous mapping,” or “ambiguously mapping” refers to a scenario when a fragment of DNA or RNA (a sequence of nucleotides) aligns with two or more locations in the target polynucleotide sequence with low confidence and/or a similar level of confidence for the two or more locations. When sequencing a genome, individual fragments are generated and then will usually be matched back to a reference genome to determine their original location. This process is known as mapping. If a read comes from a unique sequence in the genome, the read can be mapped unambiguously. However, if the read is derived from a sequence that is, for example, repeated in the genome, a mapping process may find multiple potential origins for the read. These multiple matching locations make it unclear where the read actually came from, hence the term “ambiguous mapping”.

As used herein, the term “alignment field” refers to a category of data within an alignment record, specifically detailing the relationship between a sequence read and a reference sequence. These alignment records are generally stored in standard formats like the Sequence Alignment/Map (SAM) file, which is widely used for storing sequence alignment data. The SAM format organizes alignment information into several predefined fields, each field representing a specific aspect of the alignment. For instance, fields such as QNAME (query name), FLAG (alignment properties), RNAME (reference sequence name), and POS (position of alignment) are standard components of an alignment record. Additional fields include MAPQ (mapping quality), indicating the confidence in the alignment, and CIGAR (Compact Idiosyncratic Gapped Alignment Report), which succinctly characterizes how the read aligns to the reference, encompassing matches, mismatches, insertions, and deletions.

As described herein, alignment fields are useful for interpreting the alignment's quality and accuracy. These fields contain information such as the precise (or approximate) starting position of the alignment on the reference sequence, the sequence of the read itself, the quality scores for each base in the read, and details about the read's mate in paired-end sequencing. For example, a CIGAR string is useful in identifying mismatches and gaps that may suggest variations between the read and the reference.

As described herein, an alignment field can also indicate an ambiguous alignment if, for example, the MAPQ score is low, which signifies that the read aligns equally well to multiple locations in the reference genome. Another indication of ambiguity can be inferred from the FLAG field, which may denote whether a read is mapped in a proper pair or not. Reads not properly paired often result from one read of a pair mapping confidently to one location while its mate maps to another, or not at all. In cases where the reference genome contains repetitive sequences, a read derived from such a region might map to several locations with similar scores, leading to ambiguous alignment. Ambiguously aligned reads may be flagged and optionally excluded from further analysis.

As used herein, the term “baseline scenario” (particularly when it involves the use of truth data sets), refers to a set of sequence data that has been validated and is used as a comparative standard for assessing the quality of sequencing efforts. The size of the sequence data may vary from a short sequence to a long sequence up to the size of a reference genome. Baseline scenarios may be generated for a section of the sequencing data set and used as a comparison for the rest of the same sequencing data set. For example, a portion of the sequencing data may be evaluated for some metric, such as sequence depth, and used to determine if the rest of the sequencing data (or a portion thereof) is abnormal and indicates some genomic variant.

Truth data sets may include sequences with known variants, including single nucleotide polymorphisms (SNPs), insertions, deletions, and other genetic features that have been verified through rigorous testing and are considered highly accurate. These truth sets may be employed as benchmarks to evaluate how well a new sequencing run can identify and replicate known genetic variations. They provide a point of comparison to determine the error rate of the new sequencing process by highlighting discrepancies between the newly sequenced data and the validated sequences.

As used herein, the term “putative” generally refers to “generally considered or reputed to be,” which implies an assumption based on some evidence, but without conclusive proof. In the context of genomics, when referring to “putative structural variants,” the term suggests that these are structural changes in the genome—such as deletions, duplications, insertions, inversions, or translocations—that have been identified as possible or likely variations from the reference genome, but have not yet been fully validated. Putative structural variants are typically identified through computational analyses of genomic data as described herein. Methods according to the disclosure can predict these variants by analyzing patterns in sequencing data that suggest deviations from the expected alignment to a reference genome. For instance, reads, or sets of linked reads, that span breakpoint junctions of an inversion, or clusters of reads that indicate a duplication, might lead to the identification of putative structural variants. However, these predictions may require further investigation to determine their validity.

As used herein, the term “threshold distance” in the context of identifying structural variants in a polynucleotide refers to a predefined maximum/minimum distance within which sequence reads must fall relative to anchor sequence reads to be considered relevant, such as, for example, relevant as part of the same structural variant event. The use of threshold distances is useful for filtering out less relevant reads when analyzing high-throughput sequencing data to detect genomic rearrangements such as deletions, insertions, duplications, inversions, or translocations.

As described above, anchor sequence reads are those that can be aligned with high confidence to a known location on the reference genome. In the vicinity of these anchor reads, other reads that do not align as straightforwardly may still be informative for variant detection if they are within a certain proximity—a threshold distance. The range of threshold distances can vary depending on the type of structural variant being investigated and the sequencing technology used. For example, for small Indels (Insertions/Deletions), the threshold distance might be quite small, often in the range of a few bases up to 50 bases, as the changes are relatively close to the anchor reads. For larger structural variants, the threshold distance may be set from a few hundred to several thousand bases. The larger the expected variant, the greater the distance that might be considered. When parts of the chromosome have been rearranged significantly, the threshold distance could be very large, spanning tens to hundreds of thousands of bases, as the reads indicating the breakpoints of such events could be far from the anchor points in the linear genome sequence.

These threshold distances may or may not be arbitrary. The thresholds may be determined based on empirical evidence and statistical models that account for the distribution of reads and the expected frequency of sequencing errors or natural genomic variation. By setting appropriate threshold distances, researchers can minimize false positives (incorrectly calling a variant where there is none) and false negatives (failing to detect an actual variant). The threshold distance as disclosed herein is a useful parameter in bioinformatics pipelines for structural variant detection, balancing sensitivity (detecting true variants) and specificity (not calling false variants).

Note that in the context of spatially linked reads, distance may refer to genomic distance or a physical distance in the flow cell. As used in the disclosure, the term distance may refer to both (e.g., a threshold distance is applied to both genomic distance and physical distance) and/or may be understood in the context to refer to one or the other type of distance.

As used herein, genomic distance refers to the number of base pairs between two points on a sequence within a genome. The genomic distance is a linear measurement that considers the sequence length alone, irrespective of a polynucleotide's three-dimensional structure. For example, if one gene starts at position 100,000 and another gene starts at position 200,000 on a chromosome, the genomic distance between them is 100,000 base pairs. As describe herein, in the context of identifying structural variants, a threshold genomic distance may be set to determine how far apart two reads can be to still be considered as potentially related to the same structural variant. If two reads are within this threshold genomic distance, they may be analyzed together to identify potential deletions, insertions, or other variants.

Similarly, the term “physical distance,” refers to the actual space between two fragments of polynucleotide on a flow cell. This distance may reflect the way DNA is fragmented on the flow cell. When applying thresholds to physical distances, researchers are often looking at the interaction between DNA segments in a three-dimensional space, such as in chromosome conformation capture experiments (e.g., Hi-C). A threshold for physical distance may be used to determine whether two DNA fragments are close enough to each other in order to have originated from the same original polynucleotide sequence.

Thresholds for both genomic and physical distances are useful for interpreting complex genomic data. For genomic distances, thresholds may be applied as described herein, in sequence alignment and variant calling processes to decide whether reads should be considered together for variant detection. For instance, in paired-end sequencing, if the distance between two reads exceeds the expected genomic distance based on the insert size, this could indicate a potential deletion or insertion.

For physical distances, thresholds are used in analyzing links between fragments of polynucleotides. Here, thresholds can help identify fragments that are spatially collocated (such as, by example, within a physical distance threshold) more or less frequently than expected versus random chance.

As used herein, the phrase “located spatially close” refers to the proximity of objects of fragments relative to each other or within a given space. In a broad sense, it means that the fragments are near each other in terms of physical distance, which can be measured in units, such as nanometers or units of distance on a flow cell. Defining what is considered “close” is context-dependent. Close may be defined by a threshold distance, which sets a cutoff for how near two points should be to be considered spatially close. Close may also refer generally to distance, such as determining how close two fragments are to each other, and not necessarily imply close proximity.

As used herein, the phrase “Spatially linked read pairs” in the context of genomic sequencing refers to pairs of DNA sequence reads that originate from the same polynucleotide sequence, and are expected to be a certain distance apart based on, for example, the size of the fragments. These read pairs are considered ‘linked’ because they would have been physically connected in the genome before the DNA is fragmented during, for example, library preparation for sequencing.

When determining which sequence reads are linked to other reads, such as anchor sequence reads, spatially linked read pairs are very useful. As described above, an anchor sequence read is a read that has been confidently mapped to a specific location on the reference genome. By looking at the spatially linked pair of a read, researchers can infer where the other fragment should map to the genome. If the second read of the pair does not map where expected (based on the known length of the DNA fragment), this may suggest the presence of a structural variant between the two reads.

Detecting structural variants, which include insertions, deletions, inversions, and translocations, often poses challenges because they inherently involve larger, more complex alterations to the genome than single-nucleotide polymorphisms (SNPs) or small indels. The high-confidence anchor reads become particularly crucial in this context. When reads are mapped to a reference genome, some may align perfectly or nearly perfectly, serving as anchor reads, while others may not align well or may align to multiple locations. These less reliably mapped reads may in fact be indicative of structural variants, and their accurate mapping often relies on the context provided by anchor reads.

For example, in the case of a deletion in the sample genome relative to the reference genome, an anchor read may align well at one end but have a ‘dangling’ other end that doesn't align anywhere in proximity. The presence of a high-confidence anchor read can provide the context needed to recognize that the ‘dangling’ end is not a sequencing error or artifact but is likely part of a structural variant. Likewise, for insertions, translocations, or inversions, anchor reads can offer the stable framework within which the unusual or less confidently mapped reads can be understood.

In paired-end sequencing, one read in the pair might serve as the anchor read while the other spans a structural variant. The anchor read assures that the pair exists in a specific region, giving bioinformaticians confidence to explore what the other read in the pair might reveal about structural changes in the genome. Tools specialized in detecting structural variants often use these anchor reads as starting points for ‘walking’ along the genome to find the boundaries of structural variants.

As used herein, the term “nucleotide sequence” is intended to refer to the order and type of nucleotide monomers in a nucleic acid polymer. A nucleotide sequence is a characteristic of a nucleic acid molecule and can be represented in any of a variety of formats including, for example, a depiction, image, electronic medium, series of symbols, series of numbers, series of letters, series of colors, etc. The information can be represented, for example, at single nucleotide resolution, at higher resolution (e.g., indicating molecular structure for nucleotide subunits) or at lower resolution (e.g., indicating chromosomal regions, such as haplotype steps). A series of “A,” “T,” “G,” and “C” letters is a well-known sequence representation for DNA that can be correlated, at single nucleotide resolution, with the actual sequence of a DNA molecule. A similar representation is used for RNA except that “T” is replaced with “U” in the series.

As used herein, the term “solid support” refers to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™ cyclic olefins, polyimides etc.), nylon, ceramics, resins, Zennor, silica or silica-based materials including silicon and modified silicon, carbon, metals, inorganic glasses, optical fiber bundles, and polymers. Particularly useful solid supports for some embodiments are located within a flow cell apparatus. Exemplary flow cells are set forth in further detail below.

As used herein, the term “flow cell” is intended to mean a chamber having a surface across which one or more fluid reagents can be flowed. Generally, a flow cell will have an ingress opening and an egress opening to facilitate flow of fluid. A flow cell can have multiple surfaces. Examples of flow cells and related fluidic systems and detection platforms that can be readily used in the methods of the present disclosure are described, for example, in Bentley et al, Nature 456:53-59 (2008), WO 04/018497; U.S. Pat. No. 7,057,026; WO 91/06678; WO 07/123744; U.S. Pat. Nos. 7,429,492; 7,211,414; 7,415,019; 7,405,281, and US 2008/0108082, each of which is incorporated herein by reference.

In many embodiments, a solid support to which nucleic acids are attached in a method set forth herein will have a continuous or monolithic surface. Thus, fragments can attach at spatially random locations wherein the distance between nearest neighbor fragments (or nearest neighbor clusters derived from the fragments) will be variable. The resulting arrays will have a variable or random spatial pattern of features. Alternatively, a solid support used in a method set forth herein can include an array of features that are present in a repeating pattern. In such embodiments, the features provide the locations to which modified nucleic acid polymers, or fragments thereof, can attach. Particularly useful repeating patterns are hexagonal patterns, rectilinear patterns, grid patterns, patterns having reflective symmetry, patterns having rotational symmetry, or the like. The features to which a modified nucleic acid polymer, or fragment thereof, attach can each have an area that is smaller than about 1 mm², 500 μm², 100 μm², 25 μm², 10 μm², 5 μm², 1 μm², 500 nm², or 100 nm². Alternatively, or additionally, each feature can have an area that is larger than about 100 nm², 250 nm², 500 nm², 1 μm², 2.5 μm², 5 μm², 10 μm², 100 μm², or 500 μm². A cluster or colony of nucleic acids that result from amplification of fragments on an array (whether patterned or spatially random) can similarly have an area that is in a range above or between an upper and lower limit selected from those exemplified above.

For embodiments that include an array of features on a surface, the features can be discrete, being separated by interstitial regions. Alternatively, some or all of the features on a surface can be abutting (i.e., not separated by interstitial regions). Whether the features are discrete or abutting, the average size of the features and/or average distance between the features can vary such that arrays can be high density, medium density or lower density. High density arrays are characterized as having features with average pitch of less than about 15 μm. Medium density arrays have average feature pitch of about 15 to 30 μm, while low density arrays have average feature pitch of greater than 30 μm. An array useful in the invention can have feature pitch of, for example, less than 100 μm, 50 μm, 10 μm, 5 μm, 1 μm or 0.5 μm. Alternatively, or additionally, the feature pitch can be, for example, greater than 0.1 μm, 0.5 μm, 1 μm, 5 μm, 10 μm, 50 μm, or 100 μm.

As used herein, the term “source” is intended to include an origin for a nucleic acid molecule, such as a tissue, cell, organelle, compartment, or organism. The term can be used to identify or distinguish an origin for a particular nucleic acid in a mixture that includes origins for several other nucleic acids. A source can be a particular organism in a metagenomic sample having several different species of organisms. In some embodiments the source will be identified as an individual origin (e.g., an individual cell or organism). Alternatively, the source can be identified as a species that encompasses several individuals of the same type in a sample (e.g., a species of bacteria or other organism in a metagenomic sample having several individual members of the species along with members of other species as well).

As used herein, the term “surface,” when used in reference to a material, is intended to mean an external part or external layer of the material. The surface can be in contact with another material such as a gas, liquid, gel, polymer, organic polymer, second surface of a similar or different material, metal, or coat. The surface, or regions thereof, can be substantially flat. The surface can have surface features such as wells, pits, channels, ridges, raised regions, pegs, posts or the like. The material can be, for example, a solid support, gel, or the like.

As an example, in some embodiments, fragments derived from a long nucleic acid molecule captured at the surface of a flow cell occur in a line across the surface of the flow cell (e.g., if the nucleic acid was stretched out prior to fragmentation or amplification) or in a cloud on the surface. Further, a physical map of the immobilized nucleic acid can then be generated. The physical map thus correlates the physical relationship of clusters after immobilized nucleic acid is amplified. Specifically, the physical map is used to calculate the probability that sequence data obtained from any two clusters are linked, as described in the incorporated materials of WO 2012/025250. Alternatively, or additionally, the physical map can be indicative of the genome of a particular organism in a metagenomic sample. In this latter case the physical map can indicate the order of sequence fragments in the organism's genome; however, the order need not be specified and instead the mere presence of two or more fragments in a common organism (or other source or origin) can be sufficient basis for a physical map that characterizes a mixed sample and one or more organisms therein.

In some embodiments, the physical map is generated by imaging the solid support to establish the location of the immobilized nucleic acid molecules across the surface. In some embodiments, the immobilized nucleic acid is imaged by adding an imaging agent to the solid support and detecting a signal from the imaging agent. In some embodiments, the imaging agent is a detectable label. Suitable detectable labels, include, but are not limited to, protons, haptens, radionuclides, enzymes, fluorescent labels, chemiluminescent labels, and/or chromogenic agents. For example, in some embodiments, the imaging agent is an intercalating dye or non-intercalating DNA binding agent. Any suitable intercalating dye or non-intercalating DNA binding agent as are known in the art can be used, including, but not limited to those set forth in U.S. 2012/0282617, which is incorporated herein by reference.

In certain embodiments, a plurality of modified nucleic acid molecules is flowed onto a flow cell comprising a plurality of nano-channels. As used herein, the term nano-channel refers to a narrow channel into which a long linear nucleic acid molecule is stretched. In some embodiments, no more than 1, 2, 3, 4, 5, 6, 7, 8, 9, 10, 11, 12, 13, 14, 15, 16, 17, 18, 19, 20, 30, 40, 50, 60 70, 80, 90, 100, 200, 400, 400, 500, 600, 700, 1400, 900 or no more than 500 individual long strands of nucleic acid are stretched across each nano-channel. In some embodiments the individual nano-channels are separated by a physical barrier that prevents individual long strands of target nucleic acid from interacting with multiple nano-channels. In some embodiments, the solid support comprises at least 10, 50, 100, 200, 500, 500, 3000, 5000, 10000, 30000, 50000, 80000 or at least 100000 nano-channels.

As used herein, the term “target,” when used in reference to a nucleic acid polymer, is intended to linguistically distinguish the nucleic acid, for example, from other nucleic acids, modified forms of the nucleic acid, fragments of the nucleic acid, and the like. Any of a variety of nucleic acids set forth herein can be identified as target nucleic acids, examples of which include genomic DNA (gDNA), messenger RNA (mRNA), copy or complimentary DNA (cDNA), and derivatives or analogs of these nucleic acids.

As used herein, the term “transposase” is intended to mean an enzyme that is capable of forming a functional complex with a transposon element-containing composition (e.g., transposons, transposon ends, transposon end compositions) and catalyzing insertion or transposition of the transposon element-containing composition into a target DNA with which it is incubated, for example, in an in vitro transposition reaction. The term can also include integrases from retrotransposons and retroviruses. Transposases, transposomes and transposome complexes are generally known to those of skill in the art, as exemplified by the disclosure of US Pat. App. Pub. No. 2010/0120098, which is incorporated herein by reference. Although many embodiments described herein refer to Tn5 transposase and/or hyperactive Tn5 transposase, it will be appreciated that any transposition system that is capable of inserting a transposon element with sufficient efficiency to tag a target nucleic acid can be used. In particular embodiments, a preferred transposition system is capable of inserting the transposon element in a random or in an almost random manner to tag the target nucleic acid. As used herein, the term “transposome” is intended to mean a transposase enzyme bound to a nucleic acid. typically, the nucleic acid is double stranded. For example, the complex can be the product of incubating a transposase enzyme with double-stranded transposon DNA under conditions that support non-covalent complex formation. Transposon DNA can include, without limitation, Tn5 DNA, a portion of Tn5 DNA, a transposon element composition, a mixture of transposon element compositions or other nucleic acids capable of interacting with a transposase such as the hyperactive Tn5 transposase.

As used herein, the term “transposon element” is intended to mean a nucleic acid molecule, or portion thereof, that includes the nucleotide sequences that form a transposome with a transposase or integrase enzyme. typically, the nucleic acid molecule is a double stranded DNA molecule. In some embodiments, a transposon element is capable of forming a functional complex with the transposase in a transposition reaction. As non-limiting examples, transposon elements can include the 19-bp outer end (“OE”) transposon end, inner end (“IE”) transposon end, or “mosaic end” (“ME”) transposon end recognized by a wild-type or mutant Tn5 transposase, or the R1 and R2 transposon end as set forth in the disclosure of US Pat. App. Pub. No. 2010/0120098, which is incorporated herein by reference. Transposon elements can comprise any nucleic acid or nucleic acid analogue suitable for forming a functional complex with the transposase or integrase enzyme in an in vitro transposition reaction. For example, the transposon end can comprise DNA, RNA, modified bases, non-natural bases, modified backbone, and can comprise nicks in one or both strands.

A standard NGS sequencing run yields millions of short sequences that are eventually mapped on a reference genome. A percentage of good-quality reads (1-5%) are discarded because of ambiguous genomic location. Increasing read length (2×500 or long-read sequencing), designing a specialized process to map reads on specific regions of the genome (targeted callers), using expensive and time-consuming library preparation (Illumina CLR), or a combination thereof may be implemented to address the need for disambiguating such reads that would normally be discarded. However, such approaches are costly, laborious, and time intensive. Spatial information (X and Y coordinates) obtained from a solid support surface) can be leveraged to identify fragments that are generated from a single long input fragment and subsequentially be used to improve mapping reads in ambiguous positions.

Various embodiments of the present disclosure may be a system, a method, and/or a computer program product at any possible technical detail level of integration. The computer program product may include a computer readable storage medium (or mediums) having computer readable program instructions thereon for causing a processor to carry out aspects of the present disclosure.

For example, the functionality described herein may be performed as software instructions are executed by, and/or in response to software instructions being executed by, one or more hardware processors and/or any other suitable computing devices. The software instructions and/or other executable code may be read from a computer readable storage medium (or mediums). Computer readable storage mediums may also be referred to herein as computer readable storage or computer readable storage devices.

The computer readable storage medium can be a tangible device that can retain and store data and/or instructions for use by an instruction execution device. The computer readable storage medium may be, for example, but is not limited to, an electronic storage device (including any volatile and/or non-volatile electronic storage devices), a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a solid state drive, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions (as also referred to herein as, for example, “code,” “instructions,” “module,” “application,” “software application,” and/or the like) for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. Computer readable program instructions may be callable from other instructions or from itself, and/or may be invoked in response to detected events or interrupts. Computer readable program instructions configured for execution on computing devices may be provided on a computer readable storage medium, and/or as a digital download (and may be originally stored in a compressed or installable format that requires installation, decompression or decryption prior to execution) that may then be stored on a computer readable storage medium. Such computer readable program instructions may be stored, partially or fully, on a memory device (e.g., a computer readable storage medium) of the executing computing device, for execution by the computing device. The computer readable program instructions may execute entirely on a user's computer (e.g., the executing computing device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or step diagrams of methods, apparatus (systems), and computer program products according to embodiments of the disclosure. It will be understood that each step of the flowchart illustrations and/or step diagrams, and combinations of steps in the flowchart illustrations and/or step diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or step diagram step or steps. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart(s) and/or step diagram(s) step or steps.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or step diagram step or steps. For example, the instructions may initially be carried on a magnetic disk or solid-state drive of a remote computer. The remote computer may load the instructions and/or modules into its dynamic memory and send the instructions over a telephone, cable, or optical line using a modem. A modem local to a server computing system may receive the data on the telephone/cable/optical line and use a converter device including the appropriate circuitry to place the data on a bus. The bus may carry the data to a memory, from which a processor may retrieve and execute the instructions. The instructions received by the memory may optionally be stored on a storage device (e.g., a solid-state drive) either before or after execution by the computer processor.

The flowchart and step diagrams in the Figures illustrate the architecture, functionality, and operation of possible implementations of systems, methods, and computer program products according to various embodiments of the present disclosure. In this regard, each step in the flowchart or step diagrams may represent a service, module, segment, or portion of instructions, which comprises one or more executable instructions for implementing the specified logical function(s). In some alternative implementations, the functions noted in the steps may occur out of the order noted in the Figures. For example, two steps shown in succession may, in fact, be executed substantially concurrently, or the steps may sometimes be executed in the reverse order, depending upon the functionality involved. In addition, certain steps may be omitted in some implementations. The methods and processes described herein are also not limited to any particular sequence, and the steps or states relating thereto can be performed in other sequences that are appropriate.

It will also be noted that each step of the step diagrams and/or flowchart illustration, and combinations of steps in the step diagrams and/or flowchart illustration, can be implemented by special purpose hardware-based systems that perform the specified functions or acts or carry out combinations of special purpose hardware and computer instructions. For example, any of the processes, methods, algorithms, elements, steps, applications, or other functionality (or portions of functionality) described in the preceding sections may be embodied in, and/or fully or partially automated via, electronic hardware such application-specific processors (e.g., application-specific integrated circuits (ASICs)), programmable processors (e.g., field programmable gate arrays (FPGAs)), application-specific circuitry, and/or the like (any of which may also combine custom hard-wired logic, logic circuits, ASICs, FPGAs, etc. with custom programming/execution of software instructions to accomplish the techniques).

Any of the above-mentioned processors, and/or devices incorporating any of the above-mentioned processors, may be referred to herein as, for example, “computers,” “computer devices,” “computing devices,” “hardware computing devices,” “hardware processors,” “processing units,” and/or the like. Computing devices of the above-embodiments may generally (but not necessarily) be controlled and/or coordinated by operating system software, such as Mac OS, iOS, Android, Chrome OS, Windows OS (e.g., Windows XP, Windows Vista, Windows 7, Windows 8, Windows 10, Windows 11, Windows Server, etc.), Windows CE, Unix, Linux, SunOS, Solaris, Blackberry OS, VxWorks, or other suitable operating systems. In other embodiments, the computing devices may be controlled by a proprietary operating system. Conventional operating systems control and schedule computer processes for execution, perform memory management, provide file system, networking, I/O services, and provide a user interface functionality, such as a graphical user interface (“GUI”), among other things.

Reference throughout the specification to “one example”, “another example”, “an example”, and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such value or sub-range were explicitly recited. For example, a range from about 2 kbp to about 20 kbp should be interpreted to include not only the explicitly recited limits of from about 2 kbp to about 20 kbp, but also to include individual values, such as about 3.5 kbp, about 8 kbp, about 18.2 kbp, etc., and sub-ranges, such as from about 5 kbp to about 10 kbp, etc. Furthermore, when “about” and/or “substantially” are/is utilized to describe a value, this is meant to encompass minor variations (up to +/−10%) from the stated value.

While several examples have been described in detail, it is to be understood that the disclosed examples may be modified. Therefore, the foregoing description is to be considered non-limiting.

While certain examples have been described, these examples have been presented by way of example only, and are not intended to limit the scope of the disclosure. Indeed, the novel methods described herein may be embodied in a variety of other forms. Furthermore, various omissions, substitutions and changes in the methods described herein may be made without departing from the spirit of the disclosure. The accompanying claims and their equivalents are intended to cover such forms or modifications as would fall within the scope and spirit of the disclosure.

Features, materials, characteristics, or groups described in conjunction with a particular aspect, or example are to be understood to be applicable to any other aspect or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations, one or more features from a claimed combination can, in some cases, be excised from the combination, and the combination may be claimed as a sub-combination or variation of a sub-combination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some examples, the actual steps taken in the processes illustrated and/or disclosed may differ from those shown in the figures. Depending on the example, certain of the steps described above may be removed or others may be added. Furthermore, the features and attributes of the specific examples disclosed above may be combined in different ways to form additional examples, all of which fall within the scope of the present disclosure.

For purposes of this disclosure, certain aspects, advantages, and novel features are described herein. Not necessarily all such advantages may be achieved in accordance with any particular example. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without user input or prompting, whether these features, elements, and/or steps are included or are to be performed in any particular example.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” represent a value, amount, or characteristic close to the stated value, amount, or characteristic that still performs a desired function or achieves a desired result.

The scope of the present disclosure is not intended to be limited by the specific disclosures of preferred examples in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A system for updating an alignment record of polynucleotide sequence reads from a target polynucleotide sequence comprising:
at least one processor; and
a non-transitory computer readable medium comprising instructions that, when executed by the at least one processor, cause the system to:
retrieve data comprising polynucleotide sequence reads and their spatial location on a sequencing substrate to determine spatially linked read pairs;
identify a first polynucleotide sequence read with an alignment field indicating that the first polynucleotide sequence read ambiguously maps to two or more locations in the target polynucleotide sequence;
map the first polynucleotide sequence read to a location in the target polynucleotide sequence when the first polynucleotide sequence read is spatially linked to a second polynucleotide sequence read having an alignment field indicating an unambiguous mapping; and
store an updated alignment field for the first polynucleotide sequence read in computer memory for the mapped nucleotide read if the first polynucleotide sequence read can be mapped to a location.

2. The system of claim 1, wherein the alignment field indicating that the first polynucleotide sequence read ambiguously maps to two or more locations comprises an alignment score.

3. The system of claim 2, wherein the alignment score is below a first threshold value.

4. The system of claim 1, wherein the alignment field indicating an unambiguous mapping comprises an alignment score.

5. The system of claim 4, wherein the alignment score is above a second threshold value.

6. The system of claim 1, wherein mapping the first polynucleotide sequence read comprises mapping the first polynucleotide sequence to a single location in the target polynucleotide sequence.

7. The system of claim 3, wherein the first threshold value is a MAPQ score of zero.

8. The system of claim 5, wherein the second threshold value is a MAPQ score of more than 10.

9. The system of claim 8, wherein the updated alignment field is a MAPQ score of more than 10.

10. The system of claim 1, wherein the alignment field indicating that the first polynucleotide sequence read ambiguously maps to two or more locations comprises a Boolean tag.

11. The system of claim 10, wherein the Boolean tag corresponds to a likelihood above a third threshold value that the polynucleotide sequence read has an alignment property selected from the group consisting of: chromosome of the first polynucleotide sequence read, position of the first polynucleotide sequence read, mapping quality of the alignment of the first polynucleotide sequence read and a tag indicating link information of the first polynucleotide sequence read.

12. The system of claim 10, wherein the Boolean tag is at least one of chromosome, position, mapping quality and a tag indicating link information.

13. The system of claim 12, wherein the link information comprises at least one of a number of links, a link quality, and alignments of the sequence reads that are linked to one another.

14. The system of claim 6, wherein instructions to map the nucleotide read to a single location are implemented during an alignment processing step.

15. The system of claim 6, wherein instructions to map the nucleotide read to a single location are implemented as an alignment post-processing step.

16. The system of claim 6, wherein the updated alignment field is mapping quality, wherein a mapping quality below a threshold mapping quality score indicates ambiguity.

17. The system of claim 16, wherein the mapping quality is proportional to the difference in read pair alignment scores between a first and a second-best scoring alignment.

18. The system of claim 6, wherein the updated alignment field is generated by an alignment process based on aligning the first and second polynucleotide sequences to a reference genome.

19. The system of claim 18, wherein the alignment process generates alignment scores for secondary alignments above a threshold alignment score.

20. The system of claim 19, wherein the updated alignment score is a percentage of the alignment score corresponding to the primary alignment.

21. The system of claim 1, wherein the updated alignment score is calculated based on a mapping quality.

22. The system of claim 1, wherein the updated alignment score is based on a linking quality score.

23. A method for improving mapping resolution for a polynucleotide sequence comprising:
retrieving data comprising polynucleotide sequence reads and their spatial location on a sequencing substrate to determine spatially linked read pairs;
identifying a first polynucleotide sequence read with an alignment field indicating that the first polynucleotide sequence read ambiguously maps to two or more locations in the target polynucleotide sequence;
mapping the first polynucleotide sequence read to a location in the target polynucleotide sequence when the first polynucleotide sequence read is spatially linked to a second polynucleotide sequence read having an alignment field indicating an unambiguous mapping; and
storing an updated alignment field for the first polynucleotide sequence read in computer memory for the mapped nucleotide read if the first polynucleotide sequence read can be mapped to a location.

24. A non-transitory computer readable medium comprising instructions that, when executed by one or more processor, cause the one or more processor to perform the method of claim 23.
